SEQUENCE LISTING (10 sequences)

SEQ ID NO: 1 - protein, 262 aa, C. difficile bacteriophage
MKVVIIPGHTLIGKGTGAVGYINESKETRILNDLIVKWLKIGGATVYTGRVDESSNHLADQCAIANKQETDLAVQIHFNSNATTSTPVGTETIYKTNNGKTYAERVNTRLATVFKDRGAKSDVRGLYWLNHTIAPAILIEVCFVDSKADTDYYVNNKDKVAKLIAEGILNKSISNSQGGGENKVYENVIVYTGDADKVAAQILHWQLKDSLIIEASSYKQGLGKKVYVVGGEANKLVKGDVVINGADRYETVKLALQEIDKL

SEQ ID NO: 2 - protein, 174 aa, artificial sequence
  truncated phage protein
MKVVIIPGHTLIGKGTGAVGYINESKETRILNDLIVKWLKIGGATVYTGRVDESSNHLADQCAIANKQETDLAVQIHFNSNATTSTPVGTETIYKTNNGKTYAERVNTRLATVFKDRGAKSDVRGLYWLNHTIAPAILIEVCFVDSKADTDYYVNNKDKVAKLIAEGILNKSIS

SEQ ID NO: 3 - protein, 88 aa, artificial sequence
  truncated phage protein
NSQGGGENKVYENVIVYTGDADKVAAQILHWQLKDSLIIEASSYKQGLGKKVYVVGGEANKLVKGDVVINGADRYETVKLALQEIDKL

SEQ ID NO: 4 - protein, 270 aa, C. difficile phage
MKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNINNEGVKQMYKHTIVYDGEVDKISATVVGWGYNDGKILICDIKDYVPGGTQNLYVVGGGACEKISSITKEKFIMIKGNDRFDTLYKALDFINR

SEQ ID NO: 5 - protein, 179 aa, artificial sequence
  truncated phage protein sequence
MKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNIN

SEQ ID NO: 6 - DNA, 49 bp, artificial sequence
  primer
ggagatatatccatgaaagtagtaataataccagggcacactttaattg

SEQ ID NO: 7 - DNA, 57 bp, artificial sequence
  primer
ctagaggatccccggttataatttatctatttcttgtaatgctaatttaacagtttc

SEQ ID NO: 8 - DNA, 38 bp, artificial sequence
  primer
ctaaacaaatctatatcataattctcaagggggagggg

SEQ ID NO: 9 - DNA, 38 bp, artificial sequence
  primer
cccctcccccttgagaattatgatatagatttgtttag

SEQ ID NO: 10 - protein, 13 aa, artificial sequence
  engineered binding domain
GAPVPYPDPLEPR

(19) United States
(12) Patent Application Publication
(10) Pub. No.: US 2018/0353575 A1
(43) Pub. Date: Dec. 13, 2018

(54) CLOSTRIDIUM DIFFICILE BACTERIOPHAGE LYSINS FOR DETECTION AND TREATMENT OF CLOSTRIDIUM DIFFICILE BACTERIA INFECTION

(71) Applicant: The Rockefeller University, New York, NY (US)

(72) Inventors: Vincent FISCHETTI, West Hempstead, NY (US); Chad EULER, New York, NY (US); Qiong WANG, New York, NY (US)

(21) Appl. No.: 15/757,819

(22) PCT Filed: Sep. 13, 2016
(86) PCT No.: PCT/US16/51495

§ 371 Date: Mar. 6, 2018

Related U.S. Application Data
(60) Provisional application No. 62/217,949, filed on Sep. 13, 2015.

(51) Int. Cl. A61K 38/47; C12N 1/06; A61P 1/00; C12N 9/24

(52) U.S. Cl. CPC A61K 38/47; C12N 1/06; C12Y 302/01017; C12N 9/2402; A61P 1/00

(57) ABSTRACT

Provided are compositions and articles of manufacture useful for the prophylactic and therapeutic amelioration and treatment of gram-positive bacteria, including bacilli, and related conditions. The compositions and methods incorporate and utilize Clostridium difficile derived bacteriophage lysins, particularly PlyCD truncations. Methods for treatment of humans and non-human mammals are provided.

### CROSS REFERENCE TO RELATED APPLICATIONS

This application claims priority to U.S. provisional patent application No. 62/217,949, filed Sep. 13, 2015, the disclosure of which is incorporated herein by reference.

### FIELD

The present disclosure relates generally to methods, compositions and articles of manufacture useful for the prophylactic and therapeutic amelioration and treatment of gram-positive bacteria, including Clostridium bacterial strains, including pathogenic and antibiotic-resistant bacteria, and related conditions. The invention relates to compositions and articles of manufacture incorporating isolated and engineered Clostridium bacteriophage lysins including PlyCD variants and truncations thereof, and to methods utilizing the lysin polypeptides and compositions.

### BACKGROUND

Clostridium difficile, a gram-positive anaerobic spore-forming bacterium, is the leading cause of hospital-acquired diarrhea and colitis in Europe and North America [1]. According to a new study released by the Centers for Disease Control and Prevention, Clostridium difficile caused almost half a million infections in the United States per year, and costs up to $4.8 billion each year in excess healthcare costs for acute-care facilities alone [2]. The pathogen's ability to exist in spore form allows it to persist in the hospital environment, with patients and healthcare workers being the reservoirs that spread the spores and contaminate hospital rooms and equipment. When the gastrointestinal tract microbiota of a patient becomes impaired or unbalanced, most often because of antibiotic treatment, Clostridium difficile spores can germinate into the vegetative form in the colon. Clostridium difficile pathogenesis occurs as a result of the production of two major exotoxins, A and B [3]. These toxins cause inflammation, tissue damage, and disruption of the mucosal barrier of the gastrointestinal tract, which can lead to more severe disease, such as pseudomembranous colitis and toxic megacolon, collectively called C. difficile-associated disease (CDAD). In the past decade, the emergence of new highly virulent strains, particularly the North American pulsed-field type 1 (NAP1/027/III) strains, have significantly increased the severity of Clostridium difficile infection (CDI), causing lengthy hospitalizations and substantial morbidity and mortality. In 2013, Clostridium difficile was classified by the US Centers for Disease Control and Prevention as the most urgent public health threat among antibiotic-resistant bacterial infections [2]. Therefore, there is an urgent need for better treatment and prevention of CDI [3-7].

The current standard treatment for CDI is the administration of the antibiotics metronidazole or vancomycin, which can be effective to some extent, but are often accompanied with treatment failures or episodes of post-treatment relapse [1, 8-10]. Data suggest that patients can have up to a 25% rate of recurrence of CDI after antimicrobial treatment of a first episode, and up to 60% recurrence following treatment for recurrent episodes; this recurrence is thought to be a direct and unfortunate consequence of antibiotic therapy, as available antibiotics deplete non-pathogenic gut bacteria which are protective against CDI [11-13]. CDI is associated with significant prolongation of hospital stays, indicating the need for a more rapid-onset therapy. Furthermore, antibiotic-resistant strains in Clostridium difficile have emerged, further complicating treatment [14,15]. Evolving therapeutics include fecal microbiota transplant (FMT), probiotic therapy, and monoclonal antibodies towards the toxins. While FMT seems to be the most effective treatment against recurrent/relapsing CDI, there is little evidence that it can have a beneficial effect in the acute phase of the infection, and may carry risks related to the specific route of administration of the FMT, particularly when dealing with a damaged colon [16,17]. Further, the act of transferring the fecal contents from one person into the next often dissuades some from this procedure. Results of probiotic therapy and monoclonal antibody treatment are either inconclusive or pending regulatory review, respectively [18,19]. Therefore, there is a great need for novel treatments which more effectively target Clostridium difficile without collateral damage to the protective commensal species.

In this regard, bacteriophage therapy has potential, and has been investigated in in vitro models [20-22]. To date, four temperate bacteriophages have been identified that are active against C. difficile, namely, ϕC2, ϕCD119, ϕCD27, and ϕCD6356 [23-27]. While bacteriophage therapy has potential, there are many limitations that may constrain their clinical application. Bacteriophage treatment often selects for resistant mutants and the viruses usually have a relatively narrow host range (sub-strain specific), which has been observed with the Clostridium difficile bacteriophages that target a subset of clinically relevant C.difficile infections [28,29]. This forces the development of a cocktail of phage for treatment, increasing the complexity of development.

Alternatively, using components of bacteriophage such as bacteriophage lytic enzymes (endolysins) to treat infections may reduce some of these constraints. Endolysins, also known as lysins, are highly evolved molecules produced by bacteriophages to digest the bacterial cell wall from the inside to release bacteriophage progeny [30]. For the past decade, phage endolysins have been investigated as novel antimicrobial agents to treat bacterial infections in a number of gram-positive species. Lysins may be isolated, engineered to alter and optimize their antimicrobial properties, and then applied to the outside of the cell; in this setting, lysins may have the capability to disrupt the bacterial cell wall and thus lyse the pathogenic bacteria. This concept of “lysis from without”, utilizing bacteriophage lysins to attack pathogens, has been demonstrated [31-38, 56]. Lysins normally consist of two domains: an N-terminal catalytic domain, which cleaves specific motifs in the peptidoglycan layer, and a C-terminal binding domain, which is involved in the recruiting and processing of the enzyme at the inner membrane. The specificity of a lysin is often attributed to the binding domain, which recognizes a cell wall feature specific to the bacteria that it targets [39].

The first and only previously disclosed Clostridium difficile phage endolysin, CD27L, was identified from phage ϕCD27, which can be induced by mitomycin C from Clostridium difficile strain NCTC 12727 [26, 57]. Recombinantly expressed CD27L is active against 32 diverse strains of Clostridium difficile, and its lytic activity was shown to be relatively specific to Clostridium difficile when tested against an extended panel of common commensal and pathogenic gastrointestinal species in vitro. Even though many animal models of Clostridium difficile infection have been developed to mimic the clinical symptoms of CDI in humans, to date, there have been no studies that evaluated a Clostridium difficile phage lysin as an alternative therapy for treating CDI in vivo [40,41]. The present disclosure is accordingly pertinent to the ongoing need for improved compositions and methods for addressing CDI and related pathogenic bacteria.

### SUMMARY

The present invention describes an amidase lysin, PlyCD. The sequence of both PlyCD and its catalytic domain, PlyCD₁₋₁₇₄, are significantly different from previously described lysins, including lysins specific for Clostridium difficile. PlyCD and PlyCD₁₋₁₇₄ were recombinantly expressed in E coli, purified and their biochemical activity characterized in vitro. Additionally, for the first time an endolysin was shown to have potential as a therapeutic tool against Clostridium difficile, using two mouse models of severe Clostridium difficile infection.

In an aspect, the present invention provides a lysin and derivatives thereof having killing activity against Clostridium difficile bacteria, as well as against C.sordellii and B.subtilis. The lysins of the present invention are capable of killing Clostridium difficile in mixed culture and in mixed infections in vivo. The invention thus contemplates treatment, decolonization, and/or decontamination of bacteria, cultures including but not limited to biological products and/or fecal derived contents/microbiota, or infections or in instances wherein more than one genus of bacteria is suspected or present. In particular, the invention contemplates treatment, decolonization, and/or decontamination of bacteria, cultures or infections or in instances wherein more than one type of Clostridium bacteria is suspected, present, or may be present.

In accordance with the present invention, bacteriophage lysins are provided which are derived from Clostridium difficile bacteriophages. A distinct and unique lysin has been isolated and characterized, particularly PlyCD, including an active truncation thereof PlyCD₁₋₁₇₄. The lysin polypeptides of the present invention are unique in demonstrating broad killing activity against Clostridium difficile bacteria. In one such aspect, the PlyCD₁₋₁₇₄ lysin is capable of killing Clostridium difficile strains and bacteria in animal models, as demonstrated herein in mice. PlyCD₁₋₁₇₄ is effective against antibiotic-resistant C. difficile. In a further embodiment, PlyCD₁₋₁₇₄ lysin is capable of reducing growth of Clostridium difficile strains and bacteria. The invention includes compositions and articles of manufacture comprising the lysin polypeptides and methods of prevention and treatment of bacterial growth, colonization and infections. Generally, full length PlyCD as disclosed herein can be used in embodiments of the disclosure, but it is preferred to use PlyCD₁₋₁₇₄ and or variants thereof for reasons that will be apparent from the description and figures herein.

In an aspect of the invention, a method is provided of killing gram-positive bacteria comprising the step of contacting the bacteria with a composition comprising an amount of an isolated lysin polypeptide effective to kill Clostridium difficile bacteria, the isolated lysin polypeptide comprising or consisting of PlyCD₁₋₁₇₄ lysin polypeptide or variants thereof.

Thus, a method is provided of killing Clostridium difficile bacteria comprising the step of contacting the bacteria with a composition comprising an amount of an isolated lysin polypeptide effective to kill the Clostridium difficile bacteria, the isolated lysin polypeptide comprising the amino acid sequence of SEQ ID NO: 2, or variants thereof having at least 80% identity, 85% identity, 90% identity, 95% identity or 99% identity to the polypeptide of SEQ ID NO: 2, and effective to kill the Clostridium difficile bacteria.

In an additional aspect of the above method, in addition to PlyCD₁₋₁₇₄, the composition further comprises an effective amount of the isolated lysin polypeptide comprising the amino acid sequence of SEQ ID NO:1, the isolated lysin polypeptide comprising the amino acid sequence of SEQ ID NO:1, or variants thereof having at least 80% identity to the polypeptide of SEQ ID NO:1 and effective to kill the Clostridium difficile bacteria.

The invention provides a method of killing Clostridium difficile bacteria comprising the step of contacting the bacteria with a composition comprising an amount of an isolated lysin polypeptide effective to kill gram-positive bacteria, the isolated lysin polypeptide comprising PlyCD₁₋₁₇₄.

In an aspect of the above methods of killing Clostridium difficile bacteria, the methods are performed in vitro, or ex vivo, or in vivo, so as to sterilize or decontaminate a solution, material or device, particularly intended for use by or in a human. In some embodiments, the methods are performed with use of an enema, or a colonoscopic infusion, or an oral capsule directly given to a patient in need thereof.

The invention provides a method for reducing a population of Clostridium difficile bacteria comprising the step of contacting the bacteria with a composition comprising an amount of an isolated polypeptide effective to kill at least a portion of the Clostridium difficile bacteria, the isolated polypeptide comprising the amino acid sequence of SEQ ID NO: 2, or variants thereof having at least 80% identity to the polypeptide of SEQ ID NO: 2, and effective to kill the Clostridium difficile bacteria. In an embodiment of this method, the composition further comprises an effective amount of the isolated lysin polypeptide comprising the amino acid sequence of SEQ ID NO:2, or variants thereof having at least 80% identity to the polypeptide SEQ ID NO:2 and effective to kill the Clostridium difficile bacteria.

The invention further provides a method for reducing a population of gram-positive bacteria comprising the step of contacting the bacteria with a composition comprising an amount of PlyCD₁₋₁₇₄ lysin polypeptide effective to kill gram-positive bacteria. In an aspect of this method, the composition comprises an effective amount of the isolated lysin polypeptide comprising the amino acid sequence of SEQ ID NO:2, or variants thereof having at least 80% identity, 85% identity, 90% identity or 95% identity to the polypeptide of SEQ ID NO:2 and effective to kill the Clostridium difficile bacteria.

In an aspect of the above methods for reducing a population of Clostridium difficile bacteria, the methods are performed in vitro or ex vivo so as to sterilize or decontaminate a solution, material or device, particularly intended for use by or in a human. Veterinary approaches are also included, such as for use with domesticated animals, including but not limited to swine, cattle and horses, and companion animals, such as felines and canines. Thus, the disclosure is broadly applicable to a variety of mammals that are susceptible to C. difficile infection.

The present invention further provides a method for treating an antibiotic-resistant Clostridium difficile infection in a human (or non-human mammal) comprising the step of administering to an individual having an antibiotic-resistant Clostridium difficile infection an effective amount of a composition comprising an isolated polypeptide comprising the amino acid sequence of SEQ ID NO: 2, or variants thereof having at least 80% identity, 85% identity, 90% identity or 95% identity to the polypeptide of SEQ ID NO: 2, and effective to kill C. difficile, whereby the number of Clostridium difficile in the human is reduced, and/or the infection is controlled.

In an aspect of this method, the composition may further comprise an effective amount of the isolated lysin polypeptide comprising the amino acid sequence of SEQ ID NO:1.

The invention additionally includes a method for treating a human subject exposed to or at risk for exposure to a pathogenic Clostridium difficile bacteria comprising the step of administering to the subject a composition comprising an amount of an isolated lysin polypeptide effective to kill the Clostridium difficile bacteria, the isolated lysin polypeptide comprising the amino acid sequence of SEQ SEQ ID NO: 2, or variants thereof having at least 80% identity, 85% identity, 90% identity or 95% identity to the polypeptide of SEQ ID NO: 2 and effective to kill the Clostridium difficile bacteria. In a particular aspect of this method, wherein the subject is exposed to or at risk of one of or one or more of Clostridium difficile bacteria. The subject may be a human. The subject may be a human adult, child, infant or fetus, or a non-human mammal.

In an aspect of the invention, a pharmaceutical composition is provided for killing Clostridium difficile bacteria comprising at least two isolated lysin polypeptides wherein the first isolated polypeptide comprises the amino acid sequence of SEQ ID NO:2 or variants thereof having at least 80% identity to the polypeptide of SEQ ID NO:2 and effective to kill the Clostridium difficile bacteria, and the second isolated polypeptide comprises the amino acid sequence of SEQ ID NO:1, the isolated lysin polypeptide comprising the amino acid sequence of SEQ ID NO:1, or variants thereof having at least 80% identity to the polypeptide of SEQ ID NO:1 and effective to kill the Clostridium difficile bacteria.

The invention includes an article of manufacture comprising a vessel containing a composition comprising an isolated polypeptide comprising or consisting of the amino acid sequence SEQ ID NO: 2, or variants thereof having at least 80% identity, 85% identity, 90% identity or 95% identity to the polypeptide of SEQ ID NO: 2 and effective to kill Clostridium difficile bacteria, and instructions for use of the composition in treatment of a patient exposed to or exhibiting symptoms consistent with exposure to Clostridium bacteria, where the instructions for use of the composition indicate a method for using the composition, the method comprising the steps of: a) identifying the patient suspected of having been exposed to Clostridium bacteria; and b) administering an effective amount of the composition to the patient.

The present invention also provides nucleic acids encoding the lysin polypeptides of the invention. Thus, nucleic acids are provided encoding Clostridium difficile PlyCD₁₋₁₇₄ and variants thereof as described further herein. The present invention also relates to a recombinant DNA molecule or cloned gene, or a degenerate variant thereof, which encodes a Clostridium difficile lysin or lysin polypeptide; preferably a nucleic acid molecule, in particular a recombinant DNA molecule or cloned gene, encoding the PlyCD₁₋₁₇₄ lysin polypeptide, or has a nucleotide sequence or is complementary such a DNA sequence.

In a further embodiment of the invention, the full DNA sequence of the recombinant DNA molecule, cloned gene, or nucleic acid sequence encoding a lysin polypeptide hereof may be operatively linked to an expression control sequence, which may be introduced into an appropriate host. The invention accordingly extends to unicellular hosts, including bacterial and yeast hosts, transformed with the nucleic acid sequence, cloned gene or recombinant DNA molecule comprising a DNA sequence encoding the present lysin polypeptide(s), and more particularly, the complete DNA sequence determined from these sequences set forth above.

The present invention contemplates several approaches for preparation of the lysin polypeptide(s), including as illustrated herein known recombinant techniques, and the invention is accordingly intended to cover such synthetic preparations within its scope. The isolation of the DNA and amino acid sequences disclosed herein facilitates the reproduction of the lysin polypeptide(s) by such recombinant techniques, and accordingly, the invention extends to expression vectors prepared from the disclosed DNA sequences for expression in host systems by recombinant DNA techniques, and to the resulting transformed hosts.

According to other features of certain embodiments of the present invention, a recombinant expression system is provided to produce biologically active lysin polypeptide(s). A process for preparation of the polypeptides, particularly one or more lysin polypeptide of the invention, is provided comprising culturing a host cell containing an expression vector encoding one or more lysin polypeptide(s) of the invention or capable of expressing a lysin polypeptide(s) of the invention, and recovering the polypeptide(s).

The diagnostic utility of the present invention extends to the use of the present lysin polypeptides in assays to screen for the presence of Clostridium difficile bacteria, to screen for the presence of susceptible Clostridium difficile bacteria, or to determine the susceptibility of bacteria to killing or lysing by a one or more lysin polypeptide(s) of the invention, including but not limited to those of SEQ ID NO: 1, SEQ ID NO: 2 and SEQ ID NO: 3, and variants thereof.

The present invention extends to the development of antibodies against the lysin polypeptide(s), or alternatively against the cleavage target of the lysin polypeptide, including naturally raised and recombinantly prepared antibodies. Such antibodies could include both polyclonal and monoclonal antibodies prepared by known genetic techniques, as well as bi-specific (chimeric) antibodies, and antibodies including other functionalities suiting them for additional diagnostic use conjunctive with their capability of modulating lysin activity.

Lysin polypeptides which are modified and are chimeric or fusion proteins, or which are labeled, are contemplated and provided herein. In a chimeric or fusion protein, the lysin polypeptide(s) of the invention may be covalently attached to an entity which may provide additional function or enhance the use or application of the lysin polypeptide(s), including for instance a tag, label, targeting moiety or ligand, a cell binding or cell recognizing motif or agent, an antibacterial agent, an antibody, an antibiotic.

Other objects and advantages will become apparent to those skilled in the art from a review of the following description, which proceeds with reference to the following illustrative drawings.

### BRIEF DESCRIPTION OF THE DRAWINGS

FIG. 1. The amino-acid sequence of PlyCD. (A) The full-sequence of PlyCD with the catalytic domain shown in italics; (B) The sequence alignment of PlyCD with a previously described C difficile lysin, CD27L [26]; (C) The sequence alignment of PlyCD catalytic domain, PlyCD₁₋₁₇₄, with the catalytic domain of CD27L (Letters symbolize amino acids; double dots and double plus signs symbolize identity; single plus signs and single dots symbolize similarity).

FIG. 2. The purification of PlyCD and PlyCD₁₋₁₇₄. (A) Gel analysis of the final purified PlyCD. Lanes: 1, Precision Dual-color protein standard (Bio-Rad); 2. Purified PlyCD. (B) Gel analysis of the final purified PlyCD₁₋₁₇₄. Lanes: 1, Precision Dual-color protein standard (Bio-Rad); 2. Purified PlyCD₁₋₁₇₄

FIG. 3. The molecular characterization of PlyCD. (a) The effect of pH on the lytic activity of PlyCD by analysis of optical density of culture read at 600 nm wavelength (OD600) over time; closed symbols being PlyCD-treated, open symbols being buffer controls; (b) The effect of different concentrations of NaCl on the lytic activity of PlyCD; (c) The substrate specificity of PlyCD amongst Clostridia. The ratios between the OD600 value of PlyCD-treated bacteria against buffer-treated were determined at 30 min (open column) and 60 min (closed column) post-application; all results are expressed as means±standard deviations (SD) from duplicate assays.

FIG. 4. The molecular characterization of PlyCD₁₋₁₇₄. (a) The effect of pH on the lytic activity of PlyCD₁₋₁₇₄, closed symbols being PlyCD₁₋₁₇₄-treated, open symbols being buffer control; (b) The effect of different NaCl concentrations on the lytic activity of PlyCD₁₋₁₇₄; (c) The effect of different KCl concentrations on the lytic activity of PlyCD₁₋₁₇₄; (d) The effect of different MgSO4 concentrations on the lytic activity of PlyCD₁₋₁₇₄, all experiments are performed using C difficile ATCC 43255, and results expressed as means±standard deviations (SD) from duplicate assays.

FIG. 5. Effect of truncation on lytic activity. (a) The comparison of the lytic activity between PlyCD₁₋₁₇₄ (open circle) and PlyCD (open square) against C. difficile, buffer control (closed triangle). Lysis assays comprised of cells incubated with 12.5 μM purified protein or PB (phosphate buffer); (b) The lytic activity of PlyCD₁₋₁₇₄ functions in a dose-dependent manner against C difficile; (c) The lytic activity of PlyCD₁₋₁₇₄ determined by the decrease in C difficile titer. All experiments were performed using C difficile ATCC 43255, and results expressed as means±standard deviations (SD) from duplicate assays.

FIG. 6. The substrate specificity of PlyCD₁₋₁₇₄. (a) The ratios between the OD600 values of PlyCD₁₋₁₇₄-treated Clostridia strains against buffer-treated were determined at 30 min (open column) and 60 min (closed column) post-reaction; (b) The comparison of CFU changes between hypervirulent clinical strains and the laboratory strain under different doses of PlyCD₁₋₁₇₄ over the course of 60 min. (c) The ratios between PlyCD₁₋₁₇₄-treated non-Clostridium strains against buffer-treated were determined at 30 min (open column) and 60 min (closed column) post-application. Results are the means±standard deviations (SD) from duplicate assays.

FIG. 7. The collaborative effect between PlyCD₁₋₁₇₄ and vancomycin. C.difficile cells (5×10⁶) were pre-treated with or without 101 μg/ml vancomycin for 20 min, then were centrifuged and subjected to increasing amounts of a low-activity batch PlyCD₁₋₁₇₄ in 50 mM PB (to approximate the local intestinal ionic environment) for 30 min (3.125 μg, 6.25 μg, 12.5 μg and 25 μg). CFU of remaining bacteria from each treatment group were counted after overnight incubation. Results are the means±standard deviations (SD) from duplicate assays.

FIG. 8. PlyCD₁₋₁₇₄ decreased C. difficile colonization of mice colons in an ex vivo treatment model. (a) The schematics of experimental design; (b and c) The decrease in C. difficile titer after 30 and 60 minutes of PlyCD₁₋₁₇₄ treatment compared to controls (b, n=11; c, n=7). After antibiotic treatment, C57BL/6 mice were fed 107 spores by gavage at day 0. At day 2 post-infection mice were euthanized and colons removed and cut into 3 mm tissue pieces, which were equally divided into 2 sealed plastic pouches containing 500 μl (b) or 250 μl (c) of reduced PB or PlyCD₁₋₁₇₄ (1 mg/ml). Tissues were then homogenized for 90 sec, incubated anaerobically for 1 hr, and plated to BHIS agar to enumerate CFU. Data from two independent experiments were combined and analyzed for statistical significance with the Student's t test. Mean±SD error bars shown for each figure.

FIG. 9. The binding of Rhodamine-labeled PlyCD to the cell wall of C difficile ATCC 43255.

FIG. 10. The expression and purification of the binding domain of PlyCD (‘PlyCD_{BD}’). (a) Gel analysis of arabinose-induced expression of Etag-labeled PlyCD_{BD}. Lanes: 1, Precision Dual-color protein standard (Bio-Rad); 2, Whole E. coli lysate without arabinose induction; 3. Whole E coli lysate with arabinose induction. (b) Western immunoblotting analysis of the expression of Etag-labeled PlyCD_{BD} via anti-Etag. Lanes: 1, Precision dual-color protein standard (Bio-Rad); 2, Whole E. coli lysate without arabinose induction; 3, Whole E. coli lysate with arabinose induction. (c) The purification of HIS-Etag-dual-labeled PlyCD_{BD} via nickel column. Lanes: 1, Precision dual-color protein standard (Bio-Rad); 2. Column flow through; 3-4, 10 mM imidazole wash; 5-6, 20 mM imidazole wash; 7-9, 50 mM imidazole wash; 10-11, 100 mM imidazole wash; 12, 500 mM imidazole wash. (d) Gel analysis of purified PlyCD. Lanes: 1, Precision dual-color protein standard (Bio-Rad); 2. Total E. coli lysate; 3. Final purified PlyCD_{BD}.

FIG. 11. The immunofluorescence of Etagged-PlyCD_{BD} on the cell wall of C. difficile. (a) The binding of Etagged-PlyCD_{BD} to C. difficile without the pretreatment of PlyCD1-174. (b) The binding of Etagged-PlyCD_{BD} to C. difficile after the pretreatment of PlyCD1-174.

FIG. 12. The effect of different CaCl₂ concentrations on the lytic activity of PlyCD₁₋₁₇₄. Solid symbols being PlyCD₁₋₁₇₄-treated, open symbols being buffer control; the experiment was performed using C. difficile ATCC 43255, and results expressed as means±standard deviations (SD) from duplicate assays.

FIG. 13. The mild lytic activity of an atypically low-activity batch of PlyCD₁₋₁₇₄ against C. difficile ATCC 43255 at different dose in 50 mM PB (pH7.0). Lysin-treated samples are in solid patterns, and control without lysin is in open circle. The results expressed as means±standard deviations (SD) from duplicate assays.

FIG. 14. PlyCD₁₋₁₇₄ protected mice from C. difficile infection. (a) The schematics of experimental design; (b) The survival rate of mice in each treatment group from one initial representative experiment. Lysin alone has no toxic effect to mice. After antibiotic treatment, C57BL/6 mice were fed 2×10⁶ spores by gavage at day 0. On day 1 and day 2 post infection, mice were administered with 250 μl of 400 μg of PlyCD₁₋₁₇₄ or PB by enema. Mice were monitored for survival for 7 days post-infection and the mice survival data plotted with a Kaplan-Meier survival curve.

FIG. 15. Graphical summary of data demonstrating that PlyCD₁₋₁₇₄ kills diverse C. difficile strains while sparing other types of bacteria found in the gut microbiota.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In accordance with the present disclosure there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames& S. J. Higgins eds. (1985)]; “Transcription And Translation” [B. D. Hames& S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The terms “truncated PlyCD”, and “PlyCD₁₋₁₇₄” are intended to include within their scope proteins specifically recited herein as well as all substantially homologous analogs, fragments or truncations, and allelic variations.

A “lytic enzyme” and “lytic polypeptide sequence” includes any bacterial cell wall lytic enzyme that kills one or more bacteria under suitable conditions and during a relevant time period. Examples of lytic enzymes include, without limitation, various amidase cell wall lytic enzymes. In certain aspects polypeptides of this disclosure can comprise a “lytic enzyme” or a “lytic polypeptide sequence” that is a component of a larger polypeptide.

A lytic enzyme is capable of specifically cleaving bonds that are present in the peptidoglycan of bacterial cells to disrupt the bacterial cell wall. Without intending to be bound by any particular concept, it is currently postulated that the bacterial cell wall peptidoglycan is highly conserved among most bacteria, and cleavage of only a few bonds may disrupt the bacterial cell wall. The bacteriophage lytic enzyme may be an amidase, although other types of enzymes are possible. Examples of lytic enzymes that cleave these bonds are various amidases such as muramidases, glucosaminidases, endopeptidases, or N-acetyl-muramoyl-L-alanine amidases. Fischetti et al reported that the C1 streptococcal phage lysin enzyme was an amidase [Proteomics 2008 January; 8(1):140-8]. Garcia et al reported that the Cpl lysin from a S. pneumoniae from a Cp-1 phage was a lysozyme [J Virol. 1987 August; 61(8):2573-80]. Caldentey and Bamford reported that a lytic enzyme from the phi 6 Pseudomonas phage was an endopeptidase, splitting the peptide bridge formed by melo-diaminopimilic acid and D-alanine [Biochim Biophys Acta. 1992 Sep. 4; 1159(1):44-50]. The E. coli T1 and T6 phage lytic enzymes are amidases as is the lytic enzyme from Listeria phage (ply) [Appl Environ Microbiol. 1996 August; 62(8):3057-60]. There are also other lytic enzymes known in the art that are capable of cleaving a bacterial cell wall.

The present disclosure comprises polypeptides that capable of killing host bacteria, for instance by having at least some cell wall lytic activity against the host bacteria. The polypeptide may have a sequence that encompasses native sequence lytic enzyme and variants thereof. The polypeptide may be isolated, such as from a bacteriophage (“phage”), or prepared by recombinant or synthetic approaches. The polypeptide may comprise a choline-binding portion at the carboxyl terminal side and may be characterized by an enzyme activity capable of cleaving cell wall peptidoglycan (such as amidase activity to act on amide bonds in the peptidoglycan) at the amino terminal side.

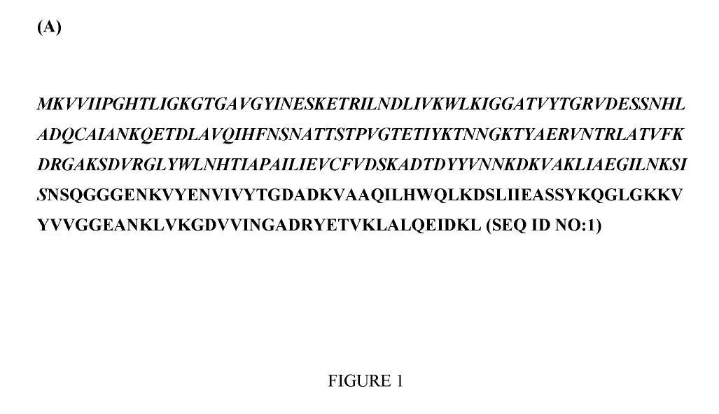

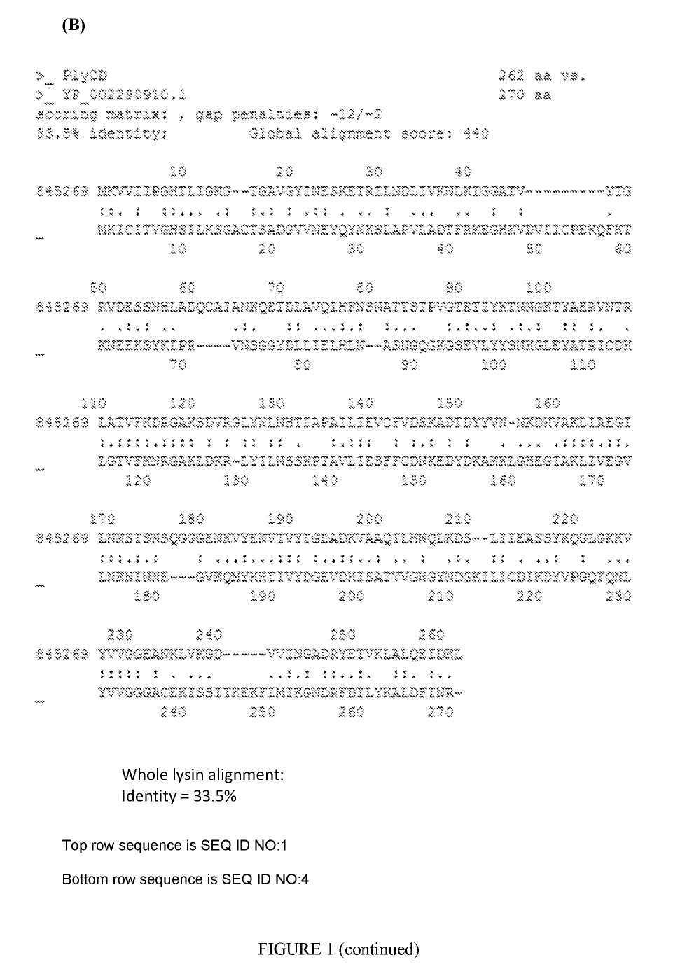

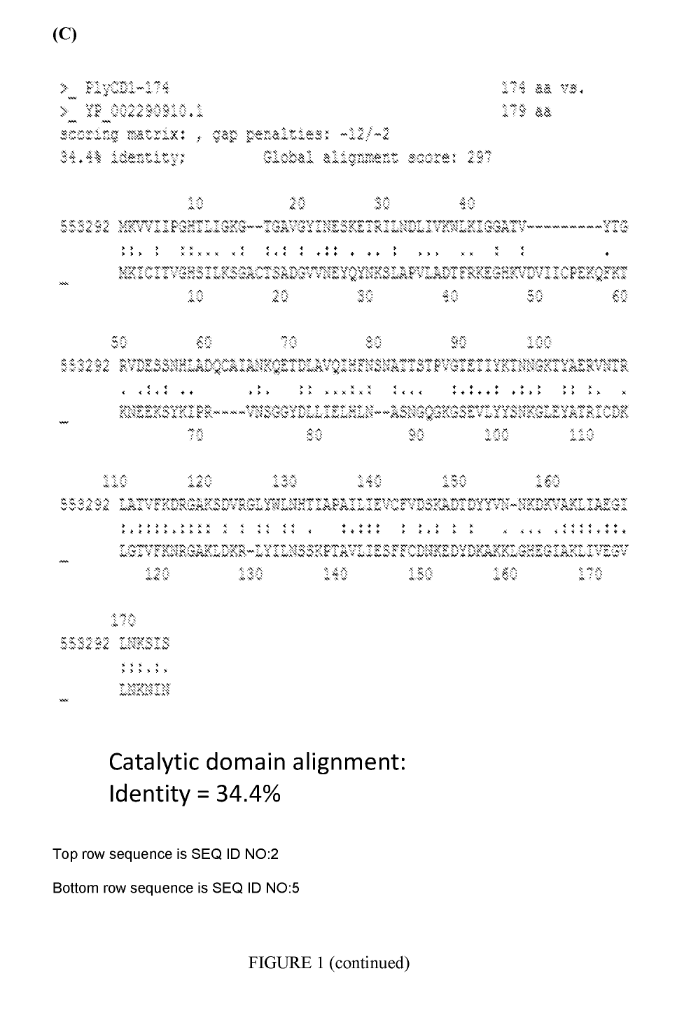

The disclosure can include lytic polypeptide sequences that are distinct from that of a naturally occurring lytic enzyme, but retain functional activity. The lytic enzyme can, in some embodiments, be genetically coded for by a bacteriophage specific for Clostridium difficile having a particular amino acid sequence identity with a segment of the lytic enzyme sequence(s) hereof, as provided in FIG. 1A, FIG. 1B, FIG. 1C, and with SEQ ID. NO. 2. For example, in some embodiments, a functionally active lytic enzyme can kill Clostridium difficile bacteria, and other susceptible bacteria as provided herein, by disrupting the cellular wall of the bacteria. A suitable polypeptide of this disclosure may have a 60, 65, 70, 75, 80, 85, 90, 95, 97, 98, 99 or 99.5% amino acid sequence identity with, for example, SEQ ID NO: 2. Such phage associated lytic enzyme variants include, for instance, lytic enzyme polypeptides wherein one or more amino acid residues are added, or deleted at the N or C terminus of the sequence of the lytic enzyme sequence(s) hereof.

Percent amino acid sequence identity with respect to the phage associated lytic enzyme sequences identified is defined herein as the percentage of amino acid residues in a polypeptide sequence of this disclosure that are identical with the amino acid residues in a phage associated lytic enzyme sequence, after aligning the sequences in the same reading frame and introducing gaps, if necessary, to achieve the maximum percent sequence identity, and not considering any conservative substitutions as part of the sequence identity.

“Percent nucleic acid sequence identity” with respect to the phage associated lytic enzyme sequences identified herein is defined as the percentage of nucleotides in a candidate sequence that are identical with the nucleotides in the phage associated lytic enzyme sequence, after aligning the sequences and introducing gaps, if necessary, to achieve the maximum percent sequence identity.

Determining the percent identity of two nucleotide or amino acid sequences can be performed using any of a variety of well-known techniques.

The present disclosure provides compositions and methods suitable for addressing CDI, as well as infection by certain other bacteria as will be further described below. The disclosure includes a characterization of a novel Clostridium difficile endolysin and a truncation thereof which comprises its catalytic domain. The truncation has strong and specific lytic activity against C. difficile. Demonstrations of the effectiveness of the truncated protein in mouse CDI models are provided, and functional differences between truncated and full length variants of the endolysin polypeptide are described and shown. In particular, it is demonstrated herein that a truncated PlyCD, namely PlyCD₁₋₁₇₄ (SEQ ID NO: 2) displays stronger lytic activity than the full-length PlyCD (SEQ ID NO: 1) which contains both the catalytic and binding domains. Further, it is demonstrated that PlyCD₁₋₁₇₄ selectivity kills C. difficile, as well as C.sordellii and B.subtilis. However, it is also demonstrated that PlyCD₁₋₁₇₄ does not selectively kill other closely related species of Clostridium other than C. difficile (i.e., it does not kill commensal non-pathogenic Clostridia), and also does not selectively kill most other types of commensal bacteria.

In particular, the PlyCD₁₋₁₇₄ showed strong lytic activity, which was almost exclusively active against C. difficile strains, both clinical and laboratory strains, compared to other strains of bacteria which may be present in the gut, such as C. septicum, C. novyi, E. faecalis, E. faecium, and L. rhamnosous. The only exceptions detected were lytic activity against B. subtilis and C. sordellii, which may indicate a very similar cell wall structure to that of C. difficile, as has been shown [Journal of Biological Chemistry 2007 May 4; 282(18):13151-9]. These results suggest that intestinal delivery of PlyCD₁₋₁₇₄ should not affect the commensal bacteria population, thus reducing potential adverse complications commonly observed following administration of available antibiotics. Accordingly, in certain aspects, the disclosure comprises compositions and methods for selectively reducing the amount of C. difficile in an individual in need thereof, without deleteriously reducing the amounts other commensal bacteria, including but not necessarily limited to C. septicum, C. novyi, E. faecalis, E. faecium, and L. rhamnosous. In certain aspects, compositions and methods of this disclosure selectively reduce the amount of C. difficile, B. subtilis, C. sordellii, or any combination thereof.

With respect to compositions and methods of this disclosure, we identified the sequence of a putative phage lysin (FIG. 1a) from a prophage in Clostridium difficile strain 630, and termed it PlyCD (Phage lysin from C. difficile). Sequence alignment of PlyCD and its catalytic domain that is comprised of amino acids 1-174 of SEQ ID NO:1 were compared with the sequence of the previously described Clostridium difficile lysin, CD27L (FIGS. 1B and 1C). There is 33% identity in amino acid sequence between PlyCD and CD27L, and a 34.6% identity between the catalytic domain of PlyCD (PlyCD₁₋₁₇₄) and CD27L.

It is noteworthy that a truncated version of CD27L (referred to as CD27L₁₋₁₇₉) has been described [Mayer M J, et al. J Bacteriol. 2011; 193(19):5477-86]. The following sequences include amino acids that denote features of the CD27L alignment:

CD27L
(SEQ ID NO: 4)
MKICITVGHS ILKSGACTSA DGVVNEYQYN KSLAPVLADT
FRKEGHKVDV IICPEKQFKT KNEEKSYKIP RVNSGGYDLL
IELHLNASNG QGKGSEVLYY SNKGLEYATR ICDKLGTVFK
NRGAKLDKRL YILNSSKPTA VLIESFFCDN KEDYDKAKKL
GHEGIAKLIV EG [symbol] EGVKQ [symbol] IVYDGEVDKI
SATVVGWGYN DGKILICDIK DYVPGGTQNL YVVGGGACEK
ISSITKEKFI MIKGNDRFDT LYKALDFINR
PlyCD
(SEQ ID NO: 1)
MKVVIIPGHT LIGKGTGAVG YINESKETRI LNDLIVKWLK
IGGATVYTGR VDESSNHLAD QCAIANKQET DLAVQIHFNS
NATTSTPVGT ETIYKTNNGK TYAERVNTRL ATVFKDRGAK
SDVRGLYWLN HTIAPAILIE VCFVDSKADT DYYVNNKDKV
AKLIAEG*ILN KSI**SNSQGGG ENK***VYENVIV YTGDADKVAA
QILHWQLKDS LIIEASSYKQ GLGKKVYVVG GEANKLVKGD
VVINGADRYE TVKLALQEID KL

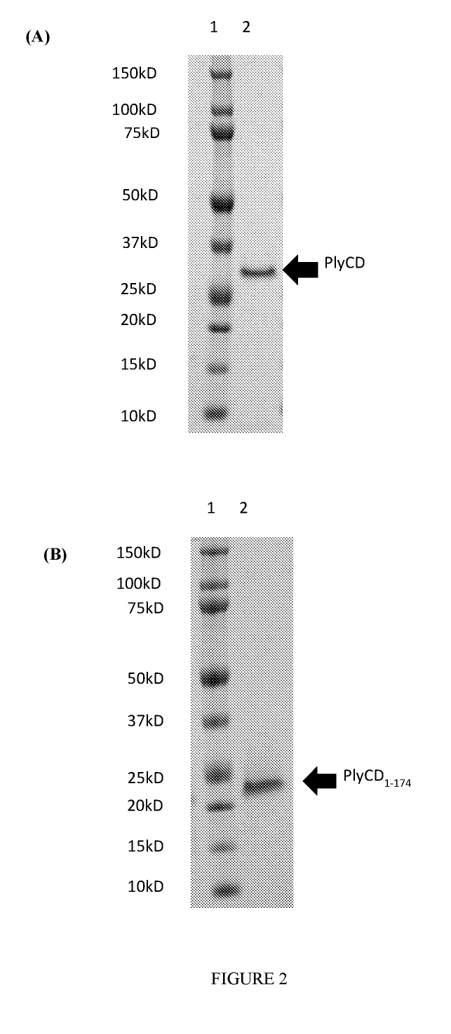

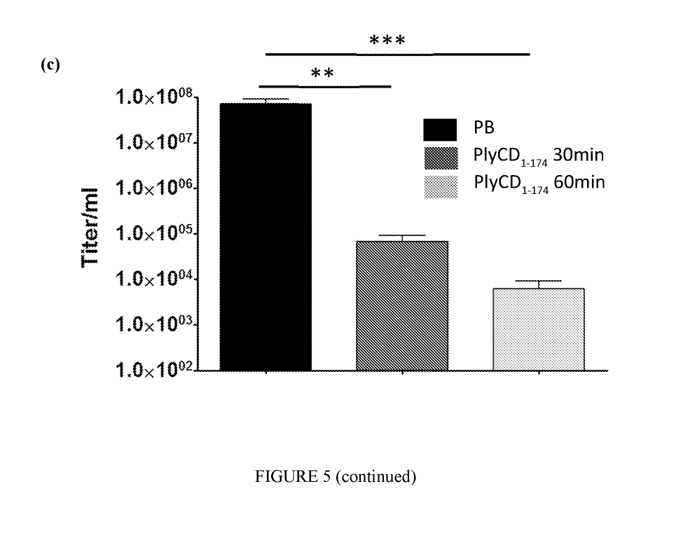

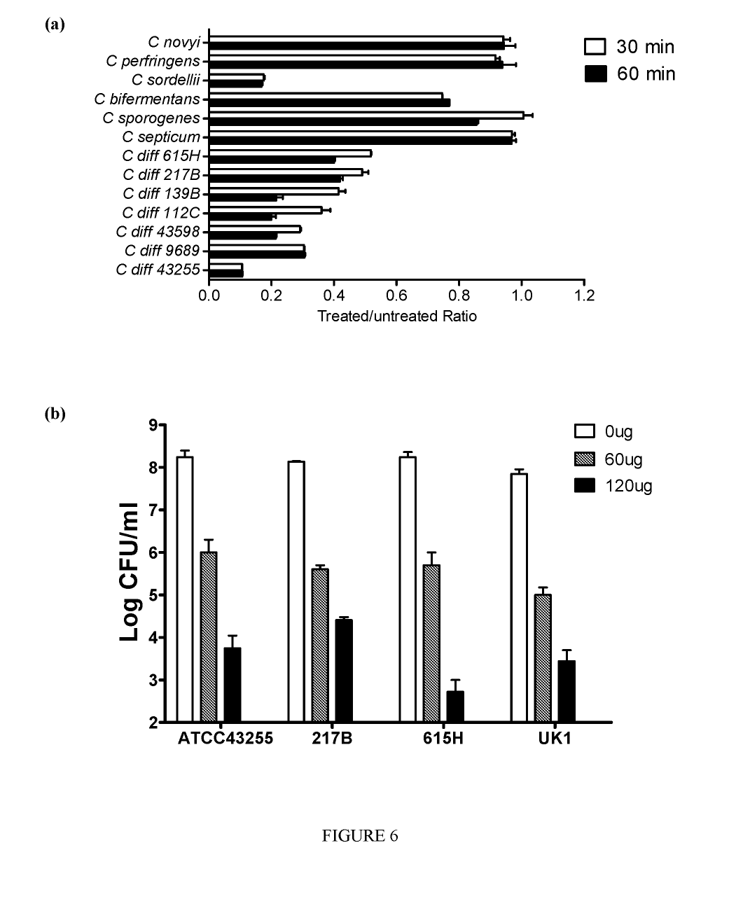

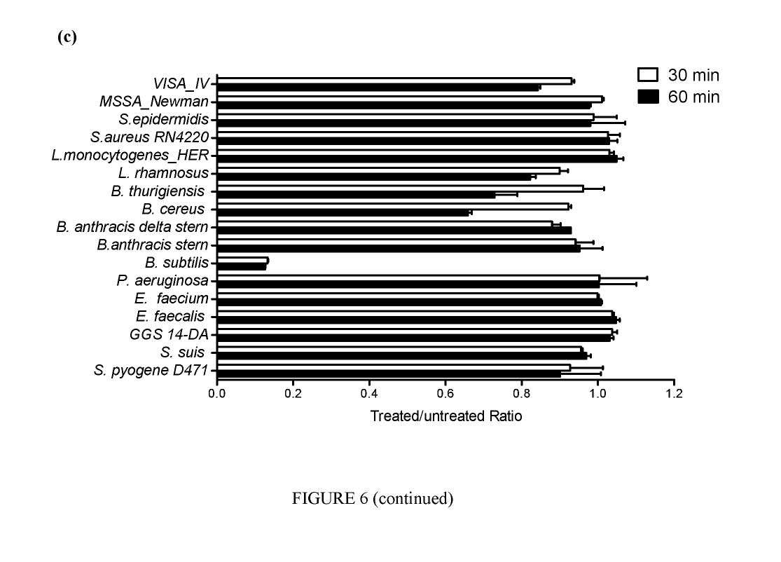

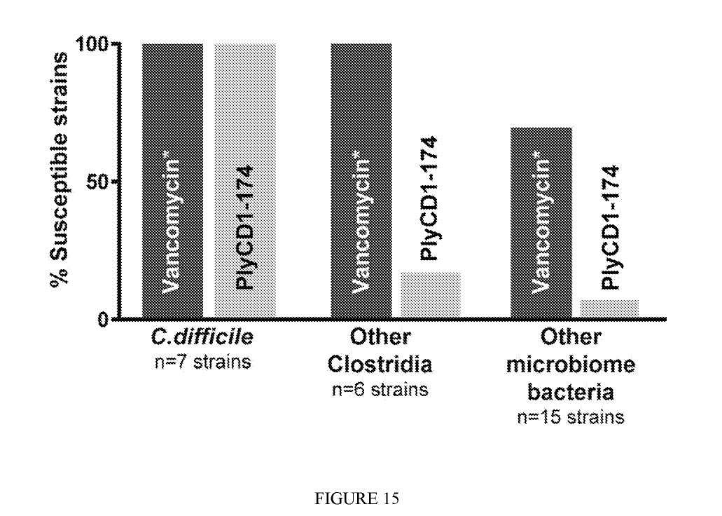

The cleavage site, denoted as the enlarged bolded “**N” at position 179 in the CD27L sequence isolates the catalytic domain from the remainder of the sequence. But in marked contrast to the selectivity of the PlyCD₁₋₁₇₄ catalytic domain relative to its full length counterpart that is demonstrated herein, the authors of Mayer et al. found no difference in the spectrum of activity for full length CD27L versus CD27L₁₋₁₇₉ across a panel of distinct organisms. Further, although there is evidence of that CD27L experiences significant autocleavage of the catalytic domain from the binding domain [Dunne M, et al. PLoS Pathog. 2014; 10(7):e1004228], it occurs at position 186, denoted as ***M in the sequence of CD27L above and in FIG. 1B, and Mayer et al. discloses that this methionine was required for autocleavage. But from a Phyre2 structural-guided sequence alignment [Kelley L A, et al., Nat Protoc. 2015; 10(6):845-58] conducted according to this disclosure, the CD27L amino acid 186 equivalent in PlyCD is at position 184, and it is the valine ***V shown in the PlyCD sequence above. In this regard, there is no evidence of naturally occurring autocleavage of PlyCD (FIG. 2A) at this Valine, and moreover, even if the aforementioned valine allowed for functional autocleavage, PlyCD cleavage equivalent to CD27L₁₋₁₇₉ would likely be PlyCD₁₋₁₈₄. Thus, there is no disclosure in Mayer et al. that would lead to making PlyCD₁₋₁₇₄ nor does Mayer et al. provide a basis for predicting the selective killing activity of PlyCD₁₋₁₇₄ for C. difficile, C. sordellii and B. subtilis, or a lack of selectivity and potency for commensal bacteria and other Clostridia strains, as shown in FIG. 6 and FIG. 15. Additionally, due to a lack of autocleavage between residues 174 and 175 there is no expectation that PlyCD₁₋₁₇₄ forms as a result of a natural process.

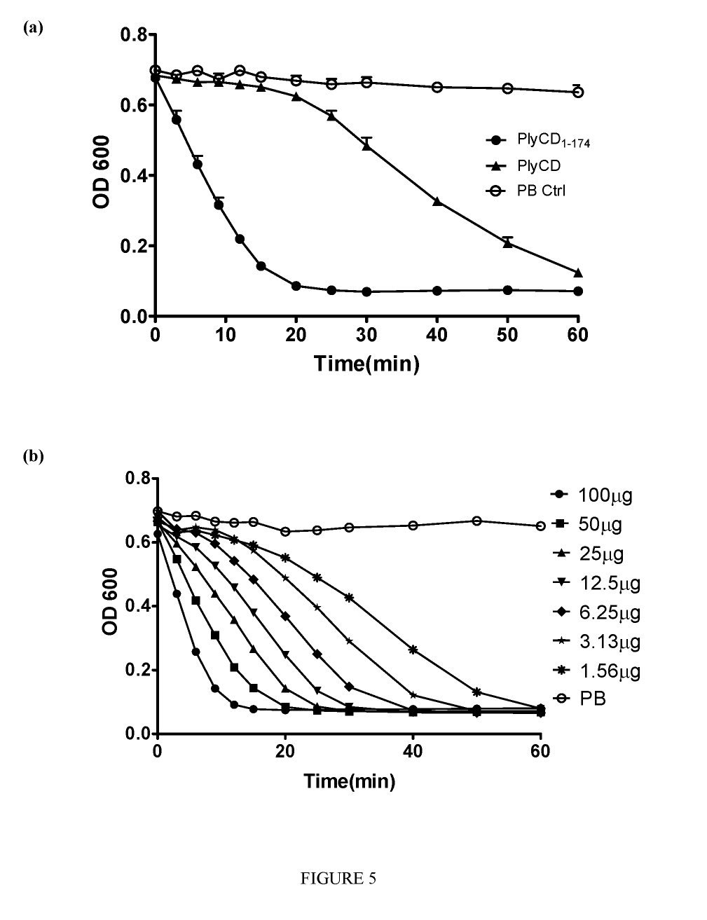

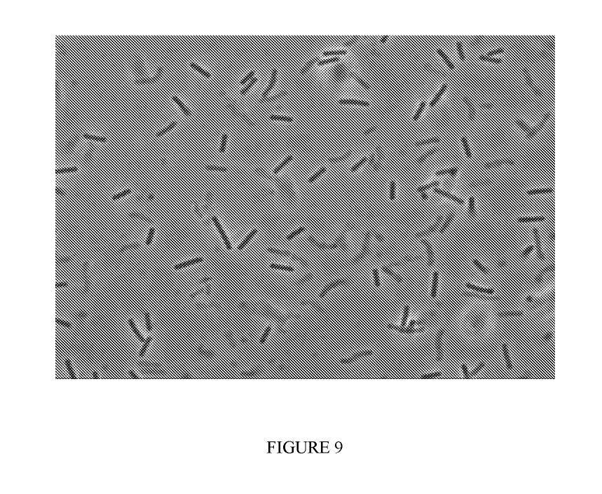

Furthermore, and without intending to be bound by any particular theory, it is believed that the role of the binding domain of a phage lysin is to bind tightly to its cell wall receptor, provide specificity, and position the catalytic domain adjacent to its substrate for cleavage. Because lysins are generated by the phage to work from the inside of a bacterial cell to release their phage progeny, structures on the outer surface of the Clostridium difficile cell wall could hinder accessibility of the peptidoglycan to the entire lysin when it is added from the outside. The presence of secondary structures on the cell surface could also affect molecules entering the wall [49]. S-layer proteins, of which Clostridium 25 difficile have several, could act as a filter preventing the larger PlyCD from entering, but not restricting its smaller catalytic domain. Other non-steric cell wall factors could reduce the ability of PlyCD to bind to the cell. In this regard, using fluorescently tagged full length PlyCD we found that PlyCD was only able to bind to cells that were degraded and not intact cells, which indicates that the binding receptor in the wall was not surface accessible (i.e., FIG. 9, showing a detection only in lysed cells). The ability of the smaller PlyCD₁₋₁₇₄ to better penetrate the cell and cleave the peptidoglycan is supported by the increased lytic activity demonstrated in FIG. 5A. Thus, the present truncation variants of PlyCD provide a unique capability to be used as exogenous agents that are uncoupled from a requirement for intracellular expression, and are accordingly suitable for use in pharmaceutical formulations that are described more fully below. Accordingly, in certain aspects, the disclosure provides a single C. difficile bacterium, and populations of C. difficile bacteria that are in physical association with polypeptides of this disclosure. In certain embodiments the disclosure comprises a population of C. difficile bacteria, and optionally C. sordellii and B. subtilis and combinations thereof, wherein the bacterial cells comprise a polypeptide of this disclosure in physical association with a component of peptidoglycan present in the bacteria. In embodiments the peptidoglycan may comprise N-deacylated glucosamine (N-deacylated NAG), N-acetylmuramic acid (NAM), N-deacylated NAM, or any combinations thereof. In one embodiment, the peptidoglycan structure is comprised of alternating NAM and NAG residues, which may be N-deacetylated, and where a tripeptide, tetrapeptide, or pentapeptide bound to the NAM residues, is crosslinked between the third amino acid of one strand to the fourth amino acid, typically a D-alanine, of a tripeptide, tetrapeptide, pentapeptide bound to a NAM residue on a neighboring strand. In a preferred embodiment, the peptidoglycan structure is comprised of alternating NAM and NAG residues, where the majority of NAG residues are N-deacetylated, and where a tripeptide, tetrapeptide, or pentapeptide bound to the NAM residues, is crosslinked between the third amino acid of one strand to the third amino acid of a tripeptide, tetrapeptide, pentapeptide bound to a NAM residue on a neighboring strand. When a tetrapeptide is present, glycine frequently occupies the 4th residue of the peptide chain. The peptidoglycan components aforementioned have been described by Peltier et al [Journal of Biological Chemistry 286(33), pp. 29053-29062, 2011].

Thus the disclosure encompasses C. difficile bacteria, and optionally C. sordellii and B. subtilis and combinations thereof, wherein a polypeptide of this disclosure has been introduced into a peptidoglycan layer of the bacteria exogenously, i.e., without being first expressed within the bacteria. The physical association between the polypeptide and peptidoglycan component can be non-covalent, and can comprise, for example, the polypeptide being adjacent to its peptidoglycan substrate such that it can perform enzymatic cleavage of the substrate, and may include cleavage intermediates, such as complexes formed between the polypeptide and the substrate during cleavage.

In certain implementations, and taking into consideration of the variability in amino acid sequence composition and polypeptide modifications as described further below, the present disclosure provides in non-limiting embodiments an isolated polypeptide and compositions comprising such polypeptides, and methods of making such polypeptides, wherein the polypeptides comprise or consist of the amino acid sequence of SEQ ID NO: 2 or an amino acid sequence with at least 95% identity to the amino acid sequence of SEQ ID NO:2, wherein the polypeptide is capable of binding specifically to and/or lysing cells of Clostridium difficile, C. sordellii and B. subtilis. In certain embodiments the polypeptides may be extended to include, for example, amino acids 176, 176, 177, 178 and 179 of SEQ ID NO:1. Thus, in embodiments, polypeptides of this disclosure may comprise contiguous amino acids identified as from 174-179 in SEQ ID NO:1, provided such segments do not include amino acids that are C-terminal to amino acid 179 of SEQ ID NO:1. In certain aspects the polypeptides used in this disclosure do not comprise the amino acid sequence of SEQ ID NO:3, which is the segment of amino acid SEQ ID NO:1 spanning amino acid 175-262. In certain aspects, the polypeptides used in this disclosure do not comprise amino acid 176, or 177, or 178 or 179 of SEQ ID NO:1, or sequences of SEQ ID NO:1 that are C-terminal to such positions.

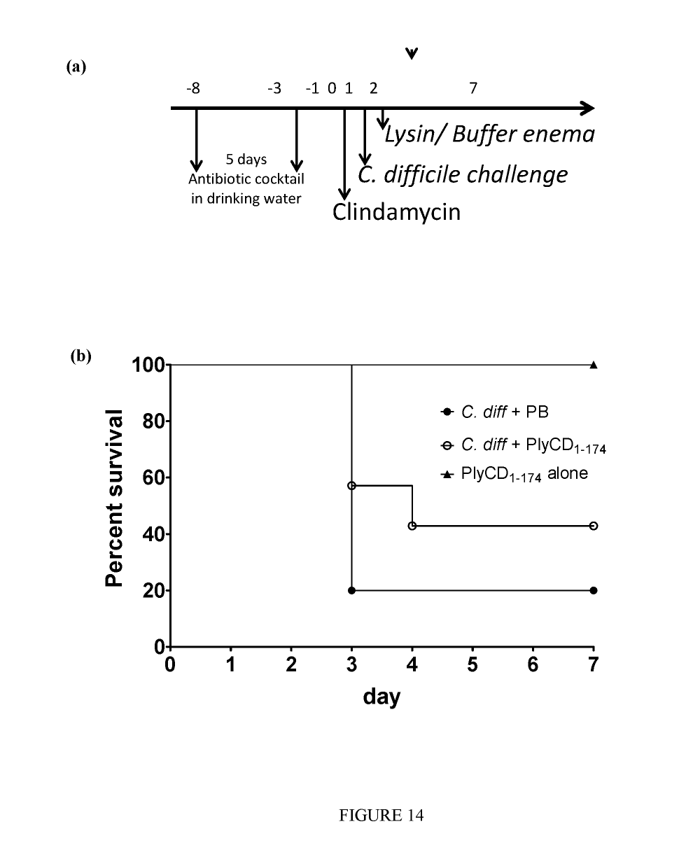

With respect to demonstrating efficacy of compositions and methods of this disclosure, the mouse infection model of CDI is believed to have relevance to human disease and to mirror many of the key features found in human infection [41, 55]. Several mouse models of CDI have been established successfully, including a gnotobiotic model [51], an antibiotic cocktail model [41], a single antibiotics model [52], and even a CDI relapse model [40]. But it is believed that this disclosure provides the first demonstration that Clostridium difficile infection in mice can be successfully treated by a bacteriophage lysin. In particular, we administered PlyCD₁₋₁₇₄, using an enema, into the colon of Clostridium difficile infected mice in an attempt to reduce mortality from CDI, as a novel route of lysin administration. The data showed that delivery of 400 μg lysin via enema was non-toxic to mice and no gross abnormal effects were observed in uninfected controls (FIG. 14B). Further analysis of Clostridium difficile infected mice showed that compared to buffer-treated controls, the lysin treated mice had an increase in survival and a delay in the rate of morbidity and mortality. It is possible that even better results could have been obtained, but were impacted by the nature of the mouse model of Clostridium difficile infection. In particular, mice were variable in the rates of Clostridium difficile disease symptoms and though some had wet perianal regions, the presence of solid stools were often found in the mouse colon upon necropsy. These stool pellets could block an even distribution of the lysin enema into the entire colon tract, thereby hindering the protective effect of lysin, and affecting the evaluation of the efficacy of PlyCD₁₋₁₇₄ in the colon. In this regard an unblocked colon tract is important to rectal delivery of drug or FMT and can be more easily achieved in human procedures, but was difficult to fully accomplish in the mice for the foregoing reasons.

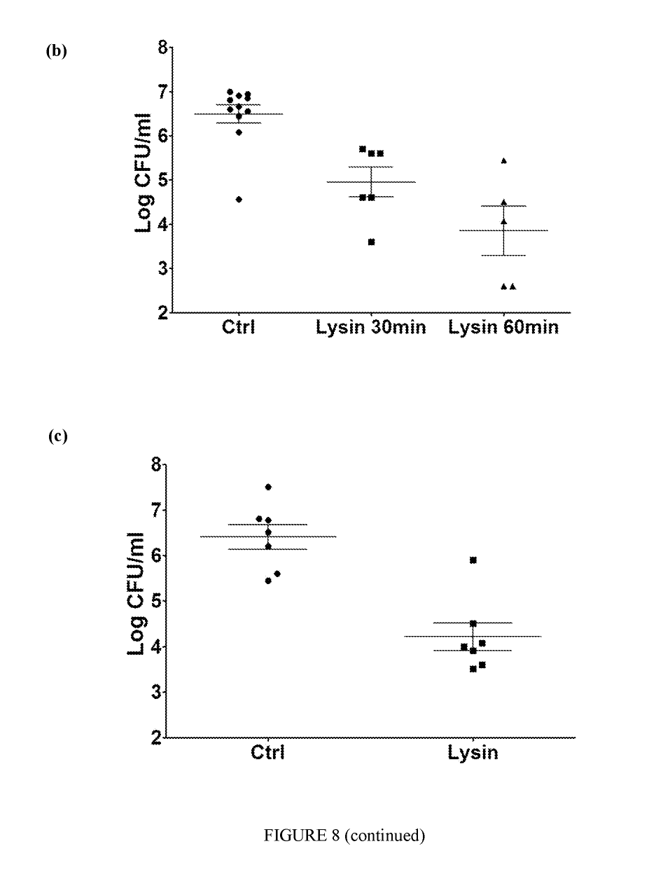

In order to address these potential impediments, we established an ex vivo model, where colons were removed from infected mice whose symptoms had progressed to the point that they had no visual solid stools (FIGS. 8A, 8B and 8C). In this model, we were able to show that the PlyCD₁₋₁₇₄ lysin works effectively in the large intestinal environment, significantly killing the Clostridium difficile organisms that were present in the colon as compared to buffer-treated controls.

Because the use of antibiotics is often unsuccessful in curing CDI, fecal microbiota transplantation (FMT) has emerged as a second-line therapy for recurrent CDI. While almost 90% successful, most transplantation failures occurred in individuals infected with the highly pathogenic NAP1/027 strain of Clostridium difficile [53]. To potentially increase the success rate of FMT by reducing residual Clostridium difficile remaining in the colon prior to administering the transplant, lysin treatment of the colon prior to delivery of the transplant may prove effective. Alternatively, since PlyCD₁₋₁₇₄ potentially has little effect on normal commensal bacteria Clostridium difficile strains, it could be combined with the donor fecal microbiota prior to the transplant, unlike conventional antibiotics which need to be cleared prior to FMT to avoid antibiotic-induced death of transplanted bacteria. The approach of removing residual Clostridium difficile prior to transplantation is supported by a randomized controlled trial of treatment of recurrent CDI [54], where colon lavage before fecal transplantation was significantly more effective (81%) at resolving CDI than without (23%) bowel lavage or after vancomycin treatment (31%). Thus, PlyCD₁₋₁₇₄ treatment could represent another tool in combating CDI, either utilizing lysin alone or in combination with other antibiotics or FMT therapies.

Polypeptides of the invention may be produced by the bacterial organism after being infected with a particular bacteriophage or other vector as either a prophylactic treatment for preventing those who have been exposed to others who have the symptoms of an infection from getting sick, or as a therapeutic treatment for those who have already become ill from the infection. In as much the lysin polypeptide sequences and nucleic acids encoding the lysin polypeptides are provided herein, the lytic enzyme(s)/polypeptide(s) may be produced via the isolated gene for the lytic enzyme from the phage genome, putting the gene into a transfer vector, and cloning said transfer vector into an expression system, using standard methods of the art, including as exemplified herein. The lytic enzyme(s) or polypeptide(s) may be truncated, chimeric, shuffled or “natural,” and may be in combination. An “altered” lytic enzyme can be produced in a number of ways. In an embodiment, a gene for the altered lytic enzyme from the phage genome is put into a transfer or movable vector, such as a plasmid, and the plasmid is cloned into an expression vector or expression system. The expression vector for producing a lysin polypeptide or enzyme of the invention may be suitable for E. coli, Bacillus, or a number of other suitable bacteria. The vector system may also be a cell free expression system. All of these methods of expressing a gene or set of genes are known in the art. The lytic enzyme may also be created by infecting Clostridium difficile with a bacteriophage specific for Clostridium difficile, wherein said at least one lytic enzyme exclusively lyses the cell wall of said Clostridium difficile having at most minimal effects on other, for example natural or commensal, bacterial flora present.

A “chimeric protein” or “fusion protein” comprises all or a biologically active part of a polypeptide of the invention operably linked to a heterologous polypeptide. Chimeric proteins or peptides are produced, for example, by combining two or more proteins having two or more active sites.

Chimeric protein and peptides can act independently on the same or different molecules, and hence have a potential to treat two or more different bacterial infections at the same time. Chimeric proteins and peptides also may be used to treat a bacterial infection by cleaving the cell wall in more than one location, thus potentially providing more rapid or effective (or synergistic) killing from a single lysin molecule or chimeric peptide.

A “heterologous” region of a DNA construct or peptide construct is an identifiable segment of DNA within a larger DNA molecule or peptide within a larger peptide molecule that is not found in association with the larger molecule in nature. An example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA or peptide as defined herein.

The term “operably linked” means that the polypeptide of the disclosure and the heterologous polypeptide are fused in-frame. The heterologous polypeptide can be fused to the N-terminus or C-terminus of the polypeptide of the disclosure. Chimeric proteins are produced enzymatically by chemical synthesis, or by recombinant DNA technology. A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetration of the proteins of either the inner membrane or the inner and outer membranes of the E. coli (FEMS Microbiol. Lett. (1998) 164(1):159-67) (incorporated herein by reference). One example of a useful fusion protein is a GST fusion protein in which the polypeptide of the disclosure is fused to the C-terminus of a GST sequence. Such a chimeric protein can facilitate the purification of a recombinant polypeptide of the disclosure.

In another embodiment, the chimeric protein or peptide contains a heterologous signal sequence at its N-terminus. For example, the native signal sequence of a polypeptide of the disclosure can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992, incorporated herein by reference). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the Protein A secretory signal (Pharmacia Biotech; Piscataway, N.J.).

The fusion protein can combine a lysin polypeptide with a protein or polypeptide of having a different capability, or providing an additional capability or added character to the lysin polypeptide. The fusion protein may be an immunoglobulin fusion protein in which all or part of a polypeptide of the disclosure is fused to sequences derived from a member of the immunoglobulin protein family. The immunoglobulin may be an antibody, for example an antibody directed to a surface protein or epitope of a susceptible or target bacteria. An immunoglobulin fusion protein can be incorporated into a pharmaceutical composition and administered to a subject to inhibit an interaction between a ligand (soluble or membrane-bound) and a protein on the surface of a cell (receptor), to thereby suppress signal transduction in vivo. The immunoglobulin fusion protein can alter bioavailability of a cognate ligand of a polypeptide of the disclosure. Inhibition of ligand/receptor interaction may be useful therapeutically, both for treating bacterial-associated diseases and disorders for modulating (i.e. promoting or inhibiting) cell survival. Moreover, an immunoglobulin fusion protein of the disclosure can be used as an immunogen to produce antibodies directed against a polypeptide of the disclosure in a subject, or to purify ligands and in screening assays to identify molecules which inhibit the interaction of receptors with ligands. Chimeric and fusion proteins and peptides of the disclosure can be produced by standard recombinant DNA techniques.

The fusion gene can be synthesized by conventional techniques. Moreover, many expression vectors are commercially available that already encode a fusion moiety (i.e., a GST polypeptide). A nucleic acid encoding a polypeptide of the invention can be cloned into such an expression vector such that the fusion moiety is linked in-frame to the polypeptide of the invention.

As used herein, shuffled proteins or peptides, gene products, or peptides for more than one related phage protein or protein peptide fragments have been randomly cleaved and reassembled into a more active or specific protein, such as a protein that is more active, for instance up to 10 to 100 fold more active than the template protein. The template protein is selected among different varieties of lysin proteins.

The modified or altered form of the protein or peptides and peptide fragments, as disclosed herein, includes protein or peptides and peptide fragments that are chemically synthesized or prepared by recombinant DNA techniques, or both.

A signal sequence of a polypeptide that may be added to a polypeptide of this disclosure can facilitate transmembrane movement of the protein and peptides and peptide fragments of the disclosure to and from mucous membranes, as well as by facilitating secretion and isolation of the secreted protein or other proteins of interest. Signal sequences are typically characterized by a core of hydrophobic amino acids which are generally cleaved from the mature protein during secretion in one or more cleavage events and are well characterized in the art, and facilitate separation, isolation and/or purification, from for example, any suitable medium by art-recognized methods. Alternatively, the signal sequence can be linked to a protein of interest using a sequence which facilitates purification, such as with a GST domain.

The disclosure also pertains to other variants of the polypeptides of the invention. Such variants can be generated by mutagenesis, i.e., discrete point mutation or truncation and can retain substantially the same, or a subset, of the biological activities of the naturally occurring form of the protein. Variants of a protein of the disclosure can be identified by screening combinatorial libraries of mutants, i.e., truncation mutants, of the protein of the disclosure. There are a variety of methods which can be used to produce libraries of potential variants of the polypeptides of the disclosure from a degenerate oligonucleotide sequence. Methods for synthesizing degenerate oligonucleotides are known in the art (see, i.e., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477, all herein incorporated by reference).

In addition, libraries of fragments of the coding sequence of a polypeptide of the disclosure can be used to generate a variegated population of polypeptides for screening and subsequent selection of variants, active fragments or truncations using a variety of techniques that are well known in the art and expression libraries can be derived which encode N-terminal and internal fragments of various sizes of the protein of interest.

Biologically active portions of a protein or peptide fragment of the embodiments, as described herein, include polypeptides comprising amino acid sequences sufficiently identical to or derived from the amino acid sequence of the phage protein of the disclosure, which include fewer amino acids than the full length protein of the phage protein and exhibit at least one activity of the corresponding full-length protein.

Homologous proteins and nucleic acids can be prepared that share functionality with such small proteins and/or nucleic acids (or protein and/or nucleic acid regions of larger molecules) as will be appreciated by a skilled artisan. Such small molecules and short regions of larger molecules that may be homologous specifically are intended as embodiments. Preferably the homology of such valuable regions is at least 50%, 65%, 75%, 80%, 85%, and preferably at least 90%, 95%, 97%, 98%, or at least 99% compared to the lysin polypeptides provided herein, including as set out in FIG. 1A, FIG. 1B, FIG. 1C and in SEQ ID. NO. 2. These percent homology values do not include alterations due to conservative amino acid substitutions.

Two amino acid sequences are “substantially homologous” when at least about 70% of the amino acid residues (preferably at least about 80%, at least about 85%, and preferably at least about 90 or 95%) are identical, or represent conservative substitutions. The sequences of comparable lysins, such as comparable PlyCD lysin and PlyCD₁₋₁₇₄ lysin, are substantially homologous when one or more, or several, or up to 10%, or up to 15%, or up to 20% of the amino acids of the lysin polypeptide are substituted with a similar or conservative amino acid substitution, and wherein the comparable lysins have the profile of activities, anti-bacterial effects, and/or bacterial specificities of a lysin, such as the PlyCD₁₋₁₇₄ lysin, disclosed herein.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

Mutations can be made in the amino acid sequences, or in the nucleic acid sequences encoding the polypeptides and lysins herein, or in active fragments or truncations thereof, such that a particular codon is changed to a codon which codes for a different amino acid, an amino acid is substituted for another amino acid, or one or more amino acids are deleted. Such a mutation is generally made by making the fewest amino acid or nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes, which do not significantly alter the activity or binding characteristics of the resulting protein.

Thus, one of skill in the art, given the benefit of the present disclosure, can make amino acid changes or substitutions in the lysin polypeptide sequence. Amino acid changes can be made to replace or substitute one or more, one or a few, one or several, one to five, one to ten, or such other number of amino acids in the sequence of the lysin(s) provided herein to generate mutants or variants thereof. Such mutants or variants thereof may be predicted for function or tested for function or capability for killing bacteria, including Clostridium bacteria, and/or for having comparable activity to the lysin(s) provided herein. Thus, changes can be made to the sequence of PlyCD₁₋₁₇₄, for example, by modifying the amino acid sequences as set out in FIG. 1A, FIG. 1B, FIG. 1C and in SEQ ID. NO. 2 hereof, and mutants or variants having a change in sequence can be tested using the assays and methods described and exemplified herein, including in the examples. One of skill in the art, on the basis of the domain structure of the lysin(s) hereof can predict one or more amino acids suitable for substitution or replacement and/or one or more amino acids which are not suitable for substitution or replacement, including reasonable conservative or non-conservative substitutions. Certain substitutions include but are not limited to: Lys for Arg and vice versa such that a positive charge may be maintained; Glu for Asp and vice versa such that a negative charge may be maintained; Ser for Thr such that a free hydroxide can be maintained; and Gln for Asn such that a free amine can be maintained. Exemplary conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa. Amino acid substitutions are typically of single residues, or can be of one or more, one or a few, one, two, three, four, five, six or seven residues; insertions usually will be on the order of about from 1 to 10 amino acid residues; and deletions will range about from 1 to 30 residues. Deletions or insertions may be in single form, but preferably are made in adjacent pairs, i.e., a deletion of 2 residues or insertion of 2 residues. Substitutions, deletions, insertions or any combination thereof may be combined to arrive at a final construct.

Substitutional variants are those in which at least one residue in the amino acid sequence has been removed and a different residue inserted in its place. Such substitutions may be made so as to generate no significant effect on the protein characteristics or when it is desired to finely modulate the characteristics of the protein.

The effects of amino acid substitutions or deletions or additions may be assessed for derivatives or variants of the lytic polypeptide(s) by analyzing the ability of the derivative or variant proteins to lyse or kill susceptible bacteria, or to complement the sensitivity to DNA cross-linking agents exhibited by phages in infected bacteria hosts.

A polypeptide or epitope thereof may be used to generate an antibody which also can be used to detect binding to the lysin or to molecules that recognize the lysin protein. The term “antibody” should be construed as covering any specific binding member or substance having a binding domain with the required specificity. Thus, this term covers antibody fragments, derivatives, functional equivalents and homologues of antibodies, including any polypeptide comprising an immunoglobulin-binding domain, whether natural or wholly or partially synthetic. Chimeric molecules comprising an immunoglobulin binding domain, or equivalent, fused to another polypeptide are therefore included. Another embodiment is a molecule such as an antibody or other specific binder that may be created through use of an epitope such as by regular immunization or by a phase display approach where an epitope can be used to screen a library if potential binders. Such molecules recognize one or more epitopes of lysin protein or a nucleic acid that encodes lysin protein.

In an embodiment the antibody or antibody fragment is in a form useful for detecting the presence of the lysin protein or, alternatively detecting the presence of a bacteria susceptible to the lysin protein. In a further embodiment the antibody may be attached or otherwise associated with the lysin polypeptide of the invention, for example in a chimeric or fusion protein, and may serve to direct the lysin to a bacterial cell or strain of interest or target. Alternatively, the lysin polypeptide may serve to direct the antibody or act in conjunction with the antibody, for example in lysing the bacterial cell wall fully or partially, so that the antibody may specifically bind to its epitope at the surface or under the surface on or in the bacteria. For example, a lysin of the invention may be attached to an anti-Streptococcal antibody and direct the antibody to its epitope.

A variety of methods for antibody synthesis are known as will be appreciated by a skilled artisan. The antibody may be conjugated (covalently complexed) with a reporter molecule or atom such as a fluorophore, an enzyme that creates an optical signal, a chemilumiphore, a microparticle, or a radioactive atom. The antibody or antibody fragment may be synthesized in vivo, after immunization of an animal, for example, the antibody or antibody fragment may be synthesized via cell culture after genetic recombination. The antibody or antibody fragment may be prepared by a combination of cell synthesis and chemical modification.

### Nucleic Acids

Nucleic acids capable of encoding the Clostridium difficile lysin polypeptide(s) of the invention are provided herein and constitute an aspect of the invention. Representative nucleic acid sequences in this context are polynucleotide sequences coding for the polypeptide of any of FIG. 1A, FIG. 1B, FIG. 1C and in SEQ ID NO: 1 and SEQ ID. NO. 2, and sequences that hybridize, under stringent conditions, with complementary sequences of the DNA sequence(s). Further variants of these sequences and sequences of nucleic acids that hybridize with those shown in the figures also are contemplated for use in production of lysing enzymes according to the disclosure, including natural variants that may be obtained. A large variety of isolated nucleic acid sequences or cDNA sequences that encode phage associated lysing enzymes and partial sequences that hybridize with such gene sequences are useful for recombinant production of the lysin enzyme(s) or polypeptide(s) of the invention.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

Within the scope of the present invention are DNA sequences encoding a lysin of the present which sequences code for a polypeptide having the same amino acid sequence as provided in FIG. 1A, FIG. 1B, FIG. 1C and in SEQ ID NO: 1 and SEQ ID. NO. 2, but which are degenerate thereto according to the genetic code. Newly derived proteins may also be selected in order to obtain variations on the characteristic of the lytic polypeptide(s).

Another feature of this invention is the expression of the DNA sequences encoding the polypeptides described herein. As is well known in the art, DNA sequences may be expressed by operatively linking them to an expression control sequence in an appropriate expression vector and employing that expression vector to transform an appropriate unicellular host. Such operative linking of a DNA sequence of this invention to an expression control sequence, of course, includes, if not already part of the DNA sequence, the provision of an initiation codon, ATG, in the correct reading frame upstream of the DNA sequence. A wide variety of host/expression vector combinations may be employed in expressing the DNA sequences of this invention.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B—W and L-M cells, African Green Monkey kidney cells (e.g., COS 1, COS 7, BSC1, BSC40, and BMT10), insect cells (e.g., Sf9), and human cells and plant cells in tissue culture.

Libraries of fragments of the coding sequence of a polypeptide can be used to generate populations, such as libraries, of polypeptides for screening and subsequent selection of variants.

Several techniques are known in the art for screening gene products of combinatorial libraries made by point mutations or truncation, and for screening cDNA libraries for gene products having a selected property.

### Compositions

The present invention provides compositions comprising bacterial lysins comprising a PlyCD₁₋₁₇₄ lysin polypeptide or variant thereof having bacterial killing activity. The invention describes, for example, exemplary PlyCD lysin truncation mutants that contain only one domain selected from the predicted amidase domain and the predicted glucosaminidase domain, for example, such a PlyCD truncation mutant includes PlyCD₁₋₁₇₄. Thus, the invention provides Clostridium difficile lysin mutants, particularly PlyCD₁₋₁₇₄ lysin mutants, which are truncated mutants containing only a catalytic domain and which exhibit improved killing activity against C. difficile, as provided and demonstrated herein. A composition is herein provided comprising a PlyCD₁₋₁₇₄ mutant lysin, having equal or greater killing activity against Clostridium cells, including Clostridium difficile compared with the full length PlyCD lysin protein, including the full length PlyCD lysin protein, the PlyCD₁₋₁₇₄ mutant lysin having a polypeptide variant of the amino acid sequence of SEQ ID NO: 2 with a modification selected from the group consisting of: a) the PlyCD mutant is a truncated mutant lysin containing only one amidase catalytic domain; b) the PlyCD₁₋₁₇₄ mutant is a truncated mutant lysin without a C-terminal binding domain; c) PlyCD has a single catalytic domain and a cell-wall binding domain; and d) the PlyCD mutant corresponds to SEQ ID NO:2, or amino acid variants thereof having one or more conservative substitutions.

Therapeutic or pharmaceutical compositions comprising the polypeptides of the invention are provided in accordance with the invention, as well as related methods of use and methods of manufacture. Therapeutic or pharmaceutical compositions may comprise one or more lytic polypeptide(s), and optionally include natural, truncated, chimeric or shuffled lytic enzymes, optionally combined with other components such as a carrier, vehicle, polypeptide, polynucleotide, holin protein(s), one or more antibiotics or suitable excipients, carriers or vehicles. The invention provides therapeutic compositions or pharmaceutical compositions of PlyCD₁₋₁₇₄, for use in the killing, alleviation, decolonization, prophylaxis or treatment of gram-positive bacteria, including bacterial infections or related conditions. The invention provides therapeutic compositions or pharmaceutical compositions of the lysins of the invention, including PlyCD₁₋₁₇₄, for use in treating, reducing or controlling contamination and/or infections by gram positive bacteria, particularly including Clostridium difficile, including in contamination or infection. Compositions are thereby contemplated and provided for therapeutic applications and local or systemic administration. Compositions comprising PlyCD₁₋₁₇₄ lysin are provided herein for use in the killing, alleviation, decolonization, prophylaxis or treatment of gram-positive bacteria, including bacterial infections or related conditions, particularly C. difficile.

The enzyme(s) or polypeptide(s) included in the therapeutic compositions may be one or more or any combination of unaltered phage associated lytic enzyme(s), truncated lytic polypeptides, variant lytic polypeptide(s), and chimeric and/or shuffled lytic enzymes. Additionally, different lytic polypeptide(s) genetically coded for by different phage for treatment of the same bacteria may be used. These lytic enzymes may also be any combination of “unaltered” lytic enzymes or polypeptides, truncated lytic polypeptide(s), variant lytic polypeptide(s), and chimeric and shuffled lytic enzymes. The lytic enzyme(s)/polypeptide(s) in a therapeutic or pharmaceutical composition for gram-positive bacteria, including Clostridium, Bacillus, Streptococcus, Staphylococcus, Enterococcus and Listeria bacteria, may be used alone or in combination with antibiotics or, if there are other invasive bacterial organisms to be treated, in combination with other phage associated lytic enzymes specific for other bacteria being targeted. The polypeptides of this disclosure may be used in conjunction with a holin protein. The amount of the holin protein may also be varied. Various antibiotics may be optionally included in the therapeutic composition with the enzyme(s) or polypeptide(s) and with or without the presence of lysostaphin. More than one lytic enzyme or polypeptide may be included in the therapeutic composition.

The pharmaceutical composition can also include a peptide or a peptide fragment of at least one lytic protein derived from the same or different bacteria species, with an optional addition of one or more complementary agent, and a pharmaceutically acceptable carrier or diluent.

The therapeutic composition may also comprise a holin protein. Holin proteins (or “holins”) are proteins which produce holes in the cell membrane. Holin proteins may form lethal membrane lesions that terminate cellular respiration in bacteria. Like the lytic proteins, holin proteins are coded for and carried by a phage [Young, et al. Trends in Microbiology v. 8, No. 4, March 2000]. Holins have been shown to be present in several bacteria (Loessner, et al., Journal of Bacteriology, August 1999, p. 4452-4460).

The pharmaceutical composition can contain a complementary agent, including one or more antimicrobial agent and/or one or more conventional antibiotics. In order to accelerate treatment of the infection, the therapeutic agent may further include at least one complementary agent which can also potentiate the bactericidal activity of the lytic enzyme. Antimicrobials act largely by interfering with the structure or function of a bacterial cell by inhibition of cell wall synthesis, inhibition of cell-membrane function and/or inhibition of metabolic functions, including protein and DNA synthesis. Antibiotics can be subgrouped broadly into those affecting cell wall peptidoglycan biosynthesis and those affecting DNA or protein synthesis in gram positive bacteria. Cell wall synthesis inhibitors, including penicillin and antibiotics like it, disrupt the rigid outer cell wall so that the relatively unsupported cell swells and eventually ruptures. Antibiotics affecting cell wall peptidoglycan biosynthesis include glycopeptides, which inhibit peptidoglycan synthesis by preventing the incorporation of N-acetylmuramic acid (NAM) and N-acetylglucosamine (NAG) peptide subunits into the peptidoglycan matrix. Available glycopeptides include vancomycin and teicoplanin. Penicillins act by inhibiting the formation of peptidoglycan cross-links. The functional group of penicillins, the β-lactam moiety, binds and inhibits D,D-transpeptidase that links the peptidoglycan molecules in bacteria. Hydrolytic enzymes continue to break down the cell wall, causing cytolysis or death due to osmotic pressure. Common penicillins include oxacillin, ampicillin and cloxacillin. Polypeptides interfere with the dephosphorylation of the C55-isoprenyl pyrophosphate, a molecule that carries peptidoglycan building-blocks outside of the plasma membrane. A cell wall-impacting polypeptide is bacitracin.

The complementary agent may be an antibiotic, such as erythromycin, clarithromycin, azithromycin, roxithromycin, other members of the macrolide family, penicillins, cephalosporins, and any combinations thereof in amounts which are effective to synergistically enhance the therapeutic effect of the lytic enzyme. Virtually any other antibiotic may be used with the altered and/or unaltered lytic enzyme. Similarly, other lytic enzymes may be included in the carrier to treat other bacterial infections. Antibiotic supplements may be used in virtually all uses of the enzyme when treating different diseases. The pharmaceutical composition can also contain a peptide or a peptide fragment of at least one lytic protein, one holin protein, or at least one holin and one lytic protein, which lytic and holin proteins are each derived from the same or different bacteria species, with an optional addition of a complementary agents, and a suitable carrier or diluent.

Also provided are compositions containing nucleic acid molecules that, either alone or in combination with other nucleic acid molecules, are capable of expressing an effective amount of a lytic polypeptide(s) or a peptide fragment of a lytic polypeptide(s) in vivo. Cell cultures containing these nucleic acid molecules, polynucleotides, and vectors carrying and expressing these molecules in vitro or in vivo, are also provided.

Therapeutic or pharmaceutical compositions may comprise lytic polypeptide(s) combined with a variety of carriers to treat the illnesses caused by the susceptible gram-positive bacteria. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as ethylenediaminetetracetic acid disodium salt (EDTA); sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl₂, CaCl₂, and others. Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% more preferably about 20%. DMSO is an aprotic solvent with a remarkable ability to enhance penetration of many locally applied drugs. DMSO may be diluted in sterile water for injection, or sodium chloride injection, or other pharmaceutically acceptable aqueous injection fluid, and used in concentrations of 0.1 to 100% (v/v). The carrier vehicle may also include Ringer's solution, a buffered solution, and dextrose solution, particularly when an intravenous solution is prepared.

Any of the carriers for the lytic polypeptide(s) may be manufactured by conventional means. However, it is preferred that any mouthwash or similar type products not contain alcohol to prevent denaturing of the polypeptide/enzyme. Similarly, when the lytic polypeptide(s) is being placed in a cough drop, gum, candy or lozenge during the manufacturing process, such placement should be made prior to the hardening of the lozenge or candy but after the cough drop or candy has cooled somewhat, to avoid heat denaturation of the enzyme.

A lytic polypeptide(s) may be added to these substances in a liquid form or in a lyophilized state, whereupon it will be solubilized when it meets body fluids such as saliva. The polypeptide(s)/enzyme may also be in a micelle or liposome.

The effective dosage rates or amounts of polypeptides of this disclosure to treat an infection will depend in part on whether the lytic enzyme/polypeptide(s) will be used therapeutically or prophylactically, the duration of exposure of the recipient to the infectious bacteria, the size and weight of the individual, etc. The duration for use of the composition containing the enzyme/polypeptide(s) also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. The concentration of the active units of enzyme believed to provide for an effective amount or dosage of enzyme may be in the range of about 100 units/ml to about 500,000 units/ml of fluid in the wet or damp environment of the nasal and oral passages, and possibly in the range of about 100 units/ml to about 50,000 units/ml. More specifically, time exposure to the active enzyme/polypeptide(s) units may influence the desired concentration of active enzyme units per ml. Carriers that are classified as “long” or “slow” release carriers (such as, for example, certain nasal sprays or lozenges) could possess or provide a lower concentration of active (enzyme) units per ml, but over a longer period of time, whereas a “short” or “fast” release carrier (such as, for example, a gargle) could possess or provide a high concentration of active (enzyme) units per ml, but over a shorter period of time. The amount of active units per mL and the duration of time of exposure depend on the nature of infection, whether treatment is to be prophylactic or therapeutic, and other variables. There are situations where it may be necessary to have a much higher unit/ml dosage or a lower unit/ml dosage.

The polypeptides may be provided in an environment having a pH, which allows for activity of the lytic enzyme/polypeptide(s). For example if a human individual has been exposed to another human with a bacterial upper respiratory disorder, the lytic enzyme/polypeptide(s) will reside in the mucosal lining and prevent and/or inhibit colonization of the infecting bacteria. Prior to, or at the time the altered lytic enzyme is put in the carrier system or oral delivery mode, the polypeptide may be provided in a stabilizing buffer environment for maintaining a pH range between about 4.0 and about 9.0, more preferably between about 5.5 and about 8.5.

A stabilizing buffer may allow for the optimum activity of the polypeptide(s). The buffer may contain a reducing reagent, such as dithiothreitol. The stabilizing buffer may also be or include a metal chelating reagent, such as EDTA, or it may also contain a phosphate or citrate-phosphate buffer, or any other buffer. The polypeptides may attack one cell wall at more than two locations, to allow the recombinant enzyme to cleave the cell wall of more than one species of bacteria, to allow the polypeptide to attack other bacteria, or any combinations thereof.

A mild surfactant can be included in a therapeutic or pharmaceutical composition in an amount effective to potentiate the therapeutic effect of the lytic enzyme/polypeptide(s) may be used in a composition. Suitable mild surfactants include, inter alia, esters of polyoxyethylenesorbitan and fatty acids (Tween series), octylphenoxypolyethoxy ethanol (Triton-X series), n-Octyl-β-D-glucopyranoside, n-Octyl-β-D-thioglucopyranoside, n-Decyl-β-D-glucopyranoside, n-Dodecyl-β-D-glucopyranoside, and biologically occurring surfactants, e.g., fatty acids, glycerides, monoglycerides, deoxycholate and esters of deoxycholate.

Preservatives may also be used in this invention and preferably comprise about 0.05% to 0.5% by weight of the total composition. The use of preservatives assures that if the product is microbially contaminated, the formulation will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidinedigluconate, or a combination thereof.

Pharmaceuticals for use in all embodiments of the invention include antimicrobial agents, anti-inflammatory agents, antiviral agents, local anesthetic agents, corticosteroids, destructive therapy agents, antifungals, and antiandrogens. In the treatment of acne, active pharmaceuticals that may be used include antimicrobial agents, especially those having anti-inflammatory properties such as dapsone, erythromycin, minocycline, tetracycline, clindamycin, and other antimicrobials. The preferred weight percentages for the antimicrobials are 0.5% to 10%.

Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is about 0.025% to 5% by weight of the total composition. Anesthetics such as benzocaine may also be used at a preferred concentration of about 2% to 25% by weight.

Corticosteroids that may be used include betamethasone dipropionate, fluocinolone actinide, betamethasone valerate, triamcinolone actinide, clobetasol propionate, desoximetasone, diflorasonediacetate, amcinonide, flurandrenolide, hydrocortisone valerate, hydrocortisone butyrate, and desonide are recommended at concentrations of about 0.01% to 1.0% by weight. Preferred concentrations for corticosteroids such as hydrocortisone or methylprednisolone acetate are from about 0.2% to about 5.0% by weight.

Additionally, the therapeutic composition may further comprise other enzymes, such as the enzyme lysostaphin for the treatment of any Staphylococcus aureus bacteria present along with the susceptible gram-positive bacteria. Mucolytic peptides, such as lysostaphin, have been suggested to be efficacious in the treatment of S. aureus infections of humans (Schaffner et al., Yale J. Biol. & Med., 39:230, 1967). Lysostaphin, a gene product of Staphylococcus simulans, exerts a bacteriostatic and bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues. Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the altered lytic enzyme, can be used in the presence or absence of antibiotics. Frequently, when a human has a bacterial infection, the infection by one genus of bacteria weakens the human body or changes the bacterial flora of the body, allowing other potentially pathogenic bacteria to infect the body. One of the bacteria that sometimes co-infects a body is Staphylococcus aureus. Many strains of Staphylococcus aureus produce penicillinase, such that Staphylococcus, Streptococcus, and other Gram positive bacterial strains will not be killed by standard antibiotics. Consequently, the use of a polypeptide of this disclosure and lysostaphin, possibly in combination with antibiotics, may serve as the most rapid and effective treatment of bacterial infections. A therapeutic composition may also include mutanolysin, and lysozyme.

Methods of application of the therapeutic composition comprising a lytic enzyme/polypeptide(s) include, but are not limited to direct, indirect, carrier and special means or any combination of approaches. Direct application of the polypeptide(s) may be by any suitable approaches to directly bring the polypeptide in contact with the site of infection or bacterial colonization, such as to the gastrointestinal tract, enemas, suppositories, tampon applications, expression by probiotics, and such. The forms in which the lytic enzyme may be administered include but are not limited to lozenges, troches, candies, injectants, chewing gums, tablets, powders, sprays, liquids, ointments, aerosols, expression directly from other microganisms, endoscopic wash, gastric lavage, or other direct injection through surgery into any part of the intestines.

When the natural and/or altered lytic enzyme(s)/polypeptide(s) is introduced directly by use of sprays, drops, ointments, enemas, washes, injections, packing, capsules and inhalers, the enzyme is in certain embodiments in a liquid or gel environment, with the liquid acting as the carrier. A dry anhydrous version of the altered enzyme may be administered by tablet, pill, capsule, inhaler or bronchial spray.

Compositions for treating infections or contaminations comprise an effective amount of at least one lytic enzyme, including PlyCD and/or PlyCD₁₋₁₇₄, according to the invention and a carrier for delivering at least one lytic enzyme to the infected or contaminated skin, coat, or external, or internal gastrointestinal surface of a companion animal or livestock. For compositions requiring absorption in the stomach and upper small intestine and/or topical delivery to these sites, particularly compositions with narrow absorption windows, bioadhesive, and/or gastroretentive drug delivery systems can be effective. Compositions requiring absorption or topical delivery only in the small intestine, enteric-coated, bioadhesive drug delivery systems can be utilized. For compositions requiring absorption or topical delivery only in the lower small intestine and colon enteric-coated, bioadhesive drug delivery systems can be utilized. Pharmaceutical compositions of the invention may be, but are not limited to powders, pellets, beads, granules, tablets, compacts, sustained release formulations, capsules, microcapsules, tablets in capsules, tablets in tablets, microspheres, shear form particles, floss, and flakes or mixtures thereof. Tablets include single layered tablets, multilayered tablets, mini tablets, bioadhesive tablets, caplets, matrix tablets, tablet within a tablet, mucoadhesive tablets. Sustained release is formulation include but are not limited to matrix type controlled release, membrane diffusion controlled release, site targeted, osmotically controlled release, pH dependent delayed release, timed release, pulsatile release, hydrodynamic balanced system; powders, pellets, beads, granules for suspension.

A composition comprising a lytic enzyme/polypeptide(s) can be administered in the form of a candy, chewing gum, lozenge, troche, tablet, capsule, a powder, an aerosol, a liquid, a liquid spray, or toothpaste for the prevention or treatment of bacterial infections associated with lower gastrointestinal illnesses. The introduction of the composition into the gastro-intestinal system can be effected by enema or colonscope, via intubation of the small bowel using for example a large bore catheter equipped with distal balloon to effect rapid passage down the jejunum, or via the oral route with enteric-coated capsules, including enteric-coated microcapsules, or via the oral route with a supplemented food or drink.

Compositions comprising polypeptides of this disclosure can be directed to the mucosal lining, where, in residence, they kill colonizing disease bacteria. The mucosal lining, as disclosed and described herein, includes, for example, the upper and lower respiratory tract, eye, buccal cavity, nose, rectum, vagina, periodontal pocket, intestines and colon. Due to natural eliminating or cleansing mechanisms of mucosal tissues, conventional dosage forms are not retained at the application site for any significant length of time.

It may be advantageous to have materials which exhibit adhesion to mucosal tissues, to be administered with one or more polypeptides and other complementary agents over a period of time. Materials having controlled release capability are particularly desirable, and the use of sustained release mucoadhesives has received a significant degree of attention. J. R. Robinson (U.S. Pat. No. 4,615,697, incorporated herein by reference) provides a good review of the various controlled release polymeric compositions used in mucosal drug delivery. The patent describes a controlled release treatment composition which includes a bioadhesive and an effective amount of a treating agent. The bioadhesive is a water swellable, but water insoluble fibrous, crosslinked, carboxy functional polymer containing (a) a plurality of repeating units of which at least about 80 percent contain at least one carboxyl functionality, and (b) about 0.05 to about 1.5 percent crosslinking agent substantially free from polyalkenyl polyether. While the polymers of Robinson are water swellable but insoluble, they are crosslinked, not thermoplastic, and are not as easy to formulate with active agents, and into the various dosage forms, as the copolymer systems of the present application. Micelles and multilamillar micelles may also be used to control the release of enzyme.

Other approaches involving mucoadhesives which are the combination of hydrophilic and hydrophobic materials, are known. Orahesive from E.R. Squibb & Co is an adhesive which is a combination of pectin, gelatin, and sodium carboxymethyl cellulose in a tacky hydrocarbon polymer, for adhering to the oral mucosa. However, such physical mixtures of hydrophilic and hydrophobic components eventually fall apart. In contrast, the hydrophilic and hydrophobic domains in this application produce an insoluble copolymer. U.S. Pat. No. 4,948,580, also incorporated by reference, describes a bioadhesive oral drug delivery system. The composition includes a freeze-dried polymer mixture formed of the copolymer poly(methyl vinyl ether/maleic anhydride) and gelatin, dispersed in an ointment base, such as mineral oil containing dispersed polyethylene. U.S. Pat. No. 5,413,792 (incorporated herein by reference) discloses paste-like preparations comprising (A) a paste-like base comprising a polyorganosiloxane and a water soluble polymeric material which are preferably present in a ratio by weight from 3:6 to 6:3, and (B) an active ingredient. U.S. Pat. No. 5,554,380 claims a solid or semisolid bioadherent orally ingestible drug delivery system containing a water-in-oil system having at least two phases. One phase comprises from about 25% to about 75% by volume of an internal hydrophilic phase and the other phase comprises from about 23% to about 75% by volume of an external hydrophobic phase, wherein the external hydrophobic phase is comprised of three components: (a) an emulsifier, (b) a glyceride ester, and (c) a wax material. U.S. Pat. No. 5,942,243 describes some representative release materials useful for administering antibacterial agents, which are incorporated by reference.

Therapeutic or pharmaceutical compositions can also contain polymeric mucoadhesives including a graft copolymer comprising a hydrophilic main chain and hydrophobic graft chains for controlled release of biologically active agents. The graft copolymer is a reaction product of (1) a polystyrene macromonomer having an ethylenically unsaturated functional group, and (2) at least one hydrophilic acidic monomer having an ethylenically unsaturated functional group. The graft chains consist essentially of polystyrene, and the main polymer chain of hydrophilic monomeric moieties, some of which have acidic functionality. The weight percent of the polystyrene macromonomer in the graft copolymer is between about 1 and about 20% and the weight percent of the total hydrophilic monomer in the graft copolymer is between 80 and 99%, and wherein at least 10% of said total hydrophilic monomer is acidic, said graft copolymer when fully hydrated having an equilibrium water content of at least 90%. Compositions containing the copolymers gradually hydrate by sorption of tissue fluids at the application site to yield a very soft jelly like mass exhibiting adhesion to the mucosal surface. During the period of time the composition is adhering to the mucosal surface, it provides sustained release of the pharmacologically active agent, which is absorbed by the mucosal tissue.

The compositions of this application may optionally contain other polymeric materials, such as poly(acrylic acid), poly,-(vinyl pyrrolidone), and sodium carboxymethyl cellulose plasticizers, and other pharmaceutically acceptable excipients in amounts that do not cause deleterious effect upon mucoadhesivity of the composition.

The dosage forms of the compositions of this invention can be prepared by conventional methods. In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. A vasoconstriction agent can be added to the formulation. The pharmaceutical preparations according to this application are provided sterile and pyrogen free.

Polypeptide(s) of the invention may also be administered by any pharmaceutically applicable or acceptable means including topically, orally or parenterally. For example, the polypeptide(s) can be administered intramuscularly, intrathecally, subdermally, subcutaneously, or intravenously to treat infections by gram-positive bacteria. In cases where parenteral injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. A vasoconstriction agent can be added to the formulation. The pharmaceutical preparations according to this application are provided sterile and pyrogen free.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The effective dosage rates or amounts of the polypeptide(s) to be administered parenterally, and the duration of treatment will depend in part on the seriousness of the infection, the weight of the patient, particularly human, the duration of exposure of the recipient to the infectious bacteria, and a variety of a number of other variables. The composition may be administered anywhere from once to several times a day, and may be administered for a short or long term period. The usage may last for days or weeks. Any dosage form employed should provide for a minimum number of units for a minimum amount of time. The concentration of the active units of enzymes believed to provide for an effective amount or dosage of enzymes may be selected as appropriate. The amount of active units per mL and the duration of time of exposure depend on the nature of infection, and the amount of contact the carrier allows the lytic enzyme(s)/polypeptide(s) to have.

### Methods and Assays

The bacterial killing capability, and indeed the significantly broad range of bacterial killing, exhibited by the lysin polypeptide(s) of the invention provides for various methods based on the antibacterial effectiveness of the polypeptide(s) of the invention. Thus, the present invention contemplates antibacterial methods, including methods for killing of gram-positive bacteria, for reducing a population of gram-positive bacteria, for treating or alleviating a bacterial infection, for treating a human subject exposed to pathogenic bacteria, and for treating a human subject at risk for such exposure. The susceptible bacteria are demonstrated herein to include the bacteria from which the phage enzyme(s) of the invention are originally derived, Clostridium difficile, as well as various other Clostridium bacterial strains. Methods of treating various conditions are also provided, including methods of prophylactic treatment of Clostridium infections, treatment of Clostridium infections, reducing Clostridium population or carriage, treating upper and lower gastrointestinal infections, treating FMTs, treating endocarditis, and treating or preventing other local or systemic infections or conditions.

The lysin(s) of the present invention demonstrate remarkable capability to kill and effectiveness against bacteria from Clostridium difficile. The invention thus contemplates treatment, decolonization, and/or decontamination of bacteria, cultures or infections or in instances wherein Clostridium difficile bacteria is suspected or present. In particular, the invention contemplates treatment, decolonization, and/or decontamination of bacteria, cultures or infections or in instances wherein Clostridium difficile bacteria is suspected, present, or may be present.

This invention also may be used to treat gastrointestinal disorders, particularly in a human. For the treatment of a gastrointestinal disorder, such as for colitis, or diarrhea, compositions can be administered via oral administration, enema, gastric gavage, endoscopic wash, and such. The concentration of the enzymes for the treatment of colitis and/or diarrhea is dependent upon the bacterial count in the subject.

The invention includes methods of treating or alleviating Clostridium, including C. difficile, related infections or conditions, including antibiotic-resistant C. difficile, particularly including wherein the bacteria or a human subject infected by or exposed to the particular bacteria, or suspected of being exposed or at risk, is contacted with or administered an amount of isolated lysin polypeptide(s) of the invention effective to kill the particular bacteria. Thus, PlyCD₁₋₁₇₄, including variations thereof, is contacted or administered so as to be effective to kill the relevant bacteria or otherwise alleviate or treat the bacterial infection.

The term ‘agent’ means any molecule, including polypeptides, antibodies, polynucleotides, chemical compounds and small molecules. In particular the term agent includes compounds such as test compounds, added additional compound(s), or lysin enzyme compounds.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term ‘prophylaxis’ is related to and encompassed in the term ‘prevention’, and refers to a measure or procedure the purpose of which is to prevent, rather than to treat or cure a disease.

‘Therapeutically effective amount’ means that amount of a polypeptide of this disclosure that will elicit the biological or medical response of a subject that is being sought by a medical doctor or other clinician. In particular, with regard to gram-positive bacterial infections and growth of gram-positive bacteria, the term “effective amount” is intended to include an effective amount of a compound or agent that will bring about a biologically meaningful decrease in the amount of or extent of infection of gram-positive bacteria, including having a bactericidal and/or bacteriostatic effect. The phrase “therapeutically effective amount” is used herein to mean an amount sufficient to prevent, and preferably reduce by at least about 30 percent, more preferably by at least 50 percent, most preferably by at least 90 percent, a clinically significant change in the growth or amount of infectious bacteria, or other feature of pathology such as for example, elevated fever or white cell count as may attend its presence and activity. Such changes can be compared to changes in any suitable reference, such as a value determined by exposure of a similar amount to bacteria other than, for example, C. difficile, C.sordellii and/or B.subtilis. Suitable controls and control values to determine, for example, relative killing activity, will be apparent to those skilled in the art given the benefit of the present disclosure.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

It is noted that in the context of treatment methods which are carried out in vivo or medical and clinical treatment methods in accordance with the present application and claims, the term subject, patient or individual is intended to refer to a human.

The terms “gram-positive bacteria”, “Gram-positive bacteria”, “gram-positive” and any variants not specifically listed, may be used herein interchangeably, and as used throughout the present application and claims refer to Gram-positive bacteria which are known and/or can be identified by the presence of certain cell wall and/or cell membrane characteristics and/or by staining with Gram stain. Gram positive bacteria are known and can readily be identified and may be selected from but are not limited to the genera Listeria, Staphylococcus, Streptococcus, Enterococcus, Mycobacterium, Corynebacterium, Bacillus and Clostridium, and include any and all recognized or unrecognized species or strains thereof. In an aspect of the invention, the PlyCD/PlyCD₁₋₁₇₄ lysin sensitive gram-positive bacteria include bacteria selected from Clostridium, Clostridium difficile.

The term “bactericidal” refers to capable of killing bacterial cells.

The term “bacteriostatic” refers to capable of inhibiting bacterial growth, including inhibiting growing bacterial cells.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

One method for treating systemic or gastrointestinal bacterial infections caused by Clostridium difficile bacteria comprises enteral treating of the infection with a therapeutic agent comprising an effective amount of one or more lysin polypeptide(s) of the invention, particularly PlyCD and/or PlyCD₁₋₁₇₄, including truncations or variants thereof, including such polypeptides as provided herein in FIG. 1A, FIG. 1B, FIG. 1C and in SEQ ID NO: 1 and SEQ ID. NO. 2 and an appropriate carrier. A number of other different methods may be used to introduce the lytic enzyme(s)/polypeptide(s). These methods include introducing the lytic enzyme(s)/polypeptide(s) orally, rectally, intravenously, intramuscularly, subcutaneously, intrathecally, and subdermally. One skilled in the art, including medical personnel, will be capable of evaluating and recognizing the most appropriate mode or means of administration, given the nature and extent of the bacterial condition and the strain or type of bacteria involved or suspected.

Infections may be also be treated by injecting into the infected tissue of the human patient a therapeutic agent comprising the appropriate lytic enzyme(s)/polypeptide(s) and a carrier for the enzyme. The carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diaminetetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils, liposomes, and ethyl oleate are also useful herein. Other phage associated lytic enzymes, along with a holin protein, may be included in the composition.

Various methods of treatment are provided for using a lytic enzyme/polypeptide(s), such as PlyCD and PlyCD₁₋₁₇₄ as exemplified herein, as a prophylactic treatment for eliminating or reducing the carriage of susceptible bacteria, preventing those humans who have been exposed to others who have the symptoms of an infection from getting sick, or as a therapeutic treatment for those who have already become ill from the infection.

The diagnostic, prophylactic and therapeutic possibilities and applications that are raised by the recognition of and isolation of the lysin polypeptide(s) of the invention, derive from the fact that the polypeptides of the invention cause direct and specific effects (e.g. killing) in susceptible bacteria. Thus, the polypeptides of the invention may be used to eliminate, characterize, or identify the relevant and susceptible bacteria.

Thus, a diagnostic method of the present invention may comprise examining a cellular sample or medium for the purpose of determining whether it contains susceptible bacteria, or whether the bacteria in the sample or medium are susceptible by means of an assay including an effective amount of one or more lysin polypeptide(s) and a means for characterizing one or more cell in the sample, or for determining whether or not cell lysis has occurred or is occurring. Patients capable of benefiting from this method include those suffering from an undetermined infection, a recognized bacterial infection, or suspected of being exposed to or carrying particular bacteria. A fluid, food, medical device, composition or other such sample, which will come in contact with a subject or patient may be examined for susceptible bacteria or may be eliminated of relevant bacteria. In one such aspect a fluid, food, medical device, composition or other such sample may be sterilized or otherwise treated to eliminate or remove any potential relevant bacteria by incubation with or exposure to one or more lytic polypeptide(s) of the invention.

The procedures and their application are all familiar to those skilled in the art and accordingly may be utilized within the scope of the present invention. In one instance, the lytic polypeptide(s) of the invention complex(es) with or otherwise binds or associates with relevant or susceptible bacteria in a sample and one member of the complex is labeled with a detectable label. The fact that a complex has formed and, if desired, the amount thereof, can be determined by known methods applicable to the detection of labels. The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

SEQ ID NO 1:
MKVVIIPGHTLIGKGTGAVGYINESKETRILNDLIVKWLKIGGATVYTGR
VDESSNHLADQCAIANKQETDLAVQIHFNSNATTSTPVGTETIYKTNNGK
TYAERVNTRLATVFKDRGAKSDVRGLYWLNHTIAPAILIEVCFVDSKADT
DYYVNNKDKVAKLIAEGILNKSISNSQGGGENKVYENVIVYTGDADKVAA
QILHWQLKDSLIIEASSYKQGLGKKVYVVGGEANKLVKGDVVINGADRYE
TVKLALQEIDKL
SEQ ID NO: 2:
MKVVIIPGHTLIGKGTGAVGYINESKETRILNDLIVKWLKIGGATVYTGR
VDESSNHLADQCAIANKQETDLAVQIHFNSNATTSTPVGTETIYKTNNGK
TYAERVNTRLATVFKDRGAKSDVRGLYWLNHTIAPAILIEVCFVDSKADT
DYYVNNKDKVAKLIAEGILNKSIS
SEQ ID NO: 3:
NSQGGGENKVYENVIVYTGDADKVAAQILHWQLKDSLIIEASSYKQGLGK
KVYVVGGEANKLVKGDVVINGADRYETVKLALQEIDKL

### Materials and Methods:

Bacterial Strains and Growth Conditions

Clostridium difficile ATCC 43255 (Ribotype 087, a high-level toxin-producing strain isolated from an abdominal wound), ATCC 9689 (Ribotype 001), ATCC 43598(Ribotype 017, isolated from infant stool) strains were obtained from ATCC. Recent clinical isolates of Clostridium difficile, strains 112C and 139B and hyper-virulent MLST2 type strains 217B and 615H was obtained from Dr. Eric Pamer (Memorial Sloan-Kettering Cancer Center, NY). C. difficile UK1 strain (Ribotype 027 was obtained from Dr. Xingmin Sun (The University of South Florida). C. novyi (VPI 2383) and C. perfringens (VPI 2641) were obtained from ATCC. C.septicum (ATCC 12464), C. sporogenes (ATCC 3584), C. bifermentans (ATCC 638), C. sordellii (ATCC 9714) were purchased from Microbiologics®. Streptococcus pyogenes D471, Group G strep 14-DA, Enterococcus faecalis V583, Enterococcus faecium EFSK2, Pseudomonas aeruginosa RS1, Streptococcus suis 6112, Bacillus subtilis SL4, B. anthracis 1659, ΔSterne, B. cereus ATCC 14579, B. thuringiensis, Lactobacillus rhamnosus ATCC 21052, Listeria monocytogenes HER 1083, Staphylococcus aureus RN4220, are part of the Rockefeller University collection. S. aureus MSSA Newman strain was obtained from Dr. Olaf Schneewind (University of Chicago, Ill.). S. aureus VISA IV was obtained from Dr. Alexander Tomasz (The Rockefeller University, NY). Staphylococcus epidermidis HER 1292 was obtained from Dr. Barry Kreiswirth (Public Health Research Institute, NJ). All strains were stored at −80° C., and cultivated at 37° C. Staphylococcus, Streptococcus, Listeria, Enterococcus, Pseudomonas, and Bacillus strains were cultivated in Difco brain heart infusion (BHI) broth (Spectrum). Lactobacillus strains were cultivated in de Mas, Rogosa, and Sharpe (MRS) broth (Sigma), Escherichia coli was grown in Luria-Bertani (LB) broth (BD Biosciences). Clostridia were cultured in BHIS media (BHI supplemented with yeast extract (0.5% (w/v), and 10% (w/v) L-cysteine), and incubated in a Whitley A35 anaerobic chamber (Microbiology International, MD), supplied with an anaerobic gas mixture (10% CO2, 85% N2, 5% H2, T. W. Smith).

Subcloning of Clostridium difficile PlyCD Gene and its Sub-Domains

The nucleotide sequence of PlyCD gene was acquired from NCBI database (NCBI Reference Sequence: YP_001088405.1), synthesized and inserted into a pUC57 vector after codon optimization for E coli expression (GenScript, NJ). After product verification by sequencing and double restrictive enzyme digestion, Hindlll and EcoRI, the PlyCD gene insert was amplified with primer sets (5′-GGAGATATATCCATGAAAGTAGTAATAATACCAGGGCACACTTTAATTG (SEQ ID NO:6), 3′-CTAGAGGATCCCCGGTTATAATTTATCTATTTCTTGTAATGCTAATTTAACAGTTTC SEQ ID NO:7), and subcloned into a pBAD24 expression vector CloneEZ PCR cloning kit (GenScript, NJ), namely pQW1. The catalytic domain of PlyCD, namely PlyCD₁₋₁₇₄ was generated by inserting a stop codon at the end of the amino-acid sequence of the catalytic domain, Cys174, via a site-directed mutagenesis kit (Agilent Technologies), using primer sets (5′-CTAAACAAATCTATATCATAATTCTCAAGGGGGAGGGG (SEQ ID NO:8), 3′-CCCCTCCCCCTTGAGAATTATGATATAGATTTGTTTAG (SEQ ID NO:9). The binding domain of PlyCD, namely PlyCDBD, was generated by replacing the N-terminal sequence (M1-Q177) with a HIS-tag followed by two E-tag (GAPVPYPDPLEPR SEQ ID NO:10) sequences in tandem. All constructs were transformed into NEB 5-F′lq competent E. coli (New England BioLabs). Positive clones were identified by colony PCR and sent for DNA sequencing (Genewiz, NJ).

Recombinant Protein Expression and Purification

After nucleotide sequence verification, the aforementioned clones were propagated in LB broth containing 100 ug/ml ampicillin until mid-log phase. The culture was then induced with 0.2% arabinose at 30° C. overnight. Cells were then pelleted, resuspended in 20 mM phosphate buffer (pH7.0) containing EDTA-free complete Mini protease inhibitor cocktail (Roche), and lysed with an EmulsiFlex C-5 homogenizer (Avestin, Ottawa, Canada). Lysate debris was removed via ultracentrifugation at 4° C. (17,000 rpm, 45 min), and the supernatant was saved and sterile filtered through a 0.2 μm filter. The target protein was then purified from the whole cell lysate by a HiTrap cation-exchange column (GE Healthcare, Uppsala, Sweden). Lysin was eluted using step-wise gradient of 0.0-1.0M NaCl in 20 mM PB (pH6.0). Extra salt was removed by filtration with a 10 kD cut-off Ultra Centricon (Amicon). The binding domain of PlyCD, PlyCDBD, was fused with a HIS-tag and dual Etags at N-terminal constructed into a pBAD25 vector, namely, pQW2. pQW2 was expressed from E. coli and purified from the whole cell lysate using a nickel column as previous described [42]. Fractions were analyzed on SDS-PAGE gels to determine the purity of the lysin in each fraction. Those with high concentrations of purified lysin were collected and buffer-exchanged against 20 mM phosphate buffer pH 7.0 (PB) via ultra centricon filtration (10 kD, EMD Millipore, MA).

Lytic Activity Assays

The lytic activity of Clostridium difficile phage lysin against Clostridium difficile strains and other bacteria were assessed based on the method previously described [43]. Basically, cells of Clostridium difficile strains were grown to mid-log phase under anaerobic conditions, and harvested by centrifugation (3,000 g, 5 min). Pellets were washed twice and resuspended with PB to generate a final OD600 of approximately 0.9. The lytic activity of lysin was calculated based on reduction in optical density (OD600) as measured in 96-well plates using a SpectraMax Plus reader (Molecular devices, Sunnyvale, Calif.). For each sample, 25 μl of either lysin (12.5 μM final concentration) or equal volume of 20 mM PB buffer was added to 180 μl of cell re-suspension. The drop of optical density at 600 nm (OD600) at 37° C. was measured once per minute for 60 min.

To study the effect of pH on the activity of lysin, the same experimental conditions and optical drop assays described above were utilized with Clostridium difficile strain ATCC 43255 and buffers of different pH, 20 mM PB (pH 6.0, 7.0, and 8.0) or 20 mM sodium acetate buffer (pH 4.0 and 5.0). To study the effect of salt on the activity of lysin, PB (pH7.0) was used that contained various concentrations of NaCl or KCl (each at 5 mM, 20 mM, 50 mM, 100 mM, or 200 mM). All experiments were performed in triplicate and results were shown as mean±SD.

Rhodamine Labeling of Lysin Constructs

NHS-Rhodamine (Thermo Scientific) was chemically linked to PlyCD and PlyCD₁₋₁₇₄ following manufacturer's instructions. The lysin construct at 1 mg was mixed with rhodamine, 10 mg/ml in DMSO, at a calculated lysin: rhodamine molar ratio of 1:10. The mixture was incubated on ice for 2 hours. Excess dye was then removed by passage through a desalting column (GE healthcare, Sweden), and fractions containing the labeled lysin construct was collected and stored at 4° C. until use.

Fluorescence Microscopy

Fluorescent microscopy procedures were adapted from a method previously described [42]. Briefly, cells from an overnight culture of Clostridium difficile strain ATCC 43255 were fixed with 2.6% paraformaldehyde in PB on ice for 45 min. After washing with 20 mM PB (pH 7.0), bacteria were fixed onto poly-L-lysine coated coverslips. Attached cells were then washed with PB, and blocked for 15 min with goat serum supplemented with 1% gelatin from cold-water fish skin (Sigma). To visualize the binding of PlyCD and PlyCD₁₋₁₇₄ to C. difficile, rhodamine-conjugated full-length PlyCD or rhodamine-conjugated PlyCD₁₋₁₇₄ was added to the slides for 10 min, and then washed with PB. To visualize the binding of PlyCD_{BD}, cells were pretreated either with or without unlabeled PlyCD₁₋₁₇₄ for 10 min, which was then washed off by PB. Recombinantly expressed PlyCD_{BD} containing dual E-tags was then added to the slides for 10 min, and washed again with PB. Following this, cells were sequentially incubated with rabbit anti-E-tag antibody (Abcam) for 1 hour at 1:500 dilution, and anti-rabbit FITC (Sigma) at 1:1000 dilution, with 3 PB washes in between. All slides were sealed with coverslips and mounting media before viewing under the microscope (Nikon Eclipse E400, Japan).

In Vivo Murine Model

The Rockefeller University's Institutional Animal Care and Use Committee approved all in vivo protocols. All experiments were conducted at The Rockefeller University's Animal Housing Facility, an AAALAC accredited research facility with all efforts to minimize suffering. All mice used in the experiments were housed in groups of 5 per cage. Autoclaved drinking water, bedding, and cages were changed every day. Chow food was radiated and kept in individual packs. Six-week-old female C57BL/6 mice were obtained from The Jackson Laboratory (Bar Harbor, Me.).

Antibiotic Administration

An antibiotic mixture of kanamycin (0.4 mg/mL), gentamycin (0.035 mg/mL), colistin (850 U/mL), metronidazole (0.215 mg/mL), and vancomycin (0.045 mg/mL) was prepared and added to the drinking water (all antibiotics were purchased from Sigma-Aldrich, St. Louis, Mo.).

Preparation of Clostridium difficile Spores

An overnight culture of Clostridium difficile strain VP110463 (ATCC 43255) was inoculated into Difco cooked meat broth (BD Diagnostic Systems, MD) and incubated at 37° C. in the anaerobic chamber. After 5 days, the meat broth culture was filtered through a sterile cell strainer (40 uM, Fisher) to remove large particles. Clostridium difficile spores in the flow through were pelleted through centrifugation (4000 rpm, 5 min). The pellet was resuspended, washed 3 times in PB, and heated at 80° C. for 30 min. Spores were then pelleted again and re-suspended in PB at a final concentration of 107 spores/ml.

Clostridium difficile Murine Infection Model

The protocol used to establish the in vivo murine CDI model was modified from a method previously described by Chen et al [41]. Mice were administered the antibiotic cocktail through drinking water for 5 days, then regular autoclaved water for 2 days. Mice then received a single dose of clindamycin (20 mg/kg) intraperitoneally 1 day before Clostridium difficile challenge. At day zero, mice were inoculated with 200 ul of 107 CFU/ml Clostridium difficile spores via gavage. Non-infected control mice were given PB via gavage instead of spores. Actual spore inoculation titers were verified by serial dilution and plating to BHIS plates containing 10% (w/v) taurocholate acid (Sigma-Aldrich, St. Louis, Mo.) for each experiment. At 24 hours and 48 hours after spore gavage, 800 μg of PlyCD₁₋₁₇₄ or PB was delivered intra-rectally to mice under ketamine/xylazine anesthesia. Intra-rectal injection was performed by inserting 20 gauge polyethylene gavage tubing (Braintree Scientific, MA) about 3.5 cm proximal to the anus. A total volume of approximately 250 μl was injected via a syringe attached to the gavage tubing. Mice were then held in a head-down vertical position for 1 min after the administration to ensure the entire volume remained in the colon. Non-infected control mice were also treated intra-rectally with 4001-g of PlyCD₁₋₁₇₄ to test the safety of the lysin. All mice were followed for 7 days, with daily monitoring for weight loss, diarrhea, morbidity and mortality. The data was analyzed by Kaplan-Meier survival curves using the Prism computer program (GraphPad Software, La Jolla, Calif.).

Clostridium difficile Ex-Vivo Colon Model

Mice were purchased and treated in the same fashion as described above, except that mice were euthanized two days after spore gavage. The colon from each mouse was then excised and cut into small (approximately 3 mm) tissue pieces. Tissue pieces from one mouse were then randomly divided into two equal sets, and placed into a plastic pouch, each containing 500 ul PB or PlyCD₁₋₁₇₄ (1 mg/ml) in PB, respectively. The two pouches containing tissue from the same mouse were processed simultaneously in a Stomacher Biomaster (Seward, UK) for 90 seconds, to ensure a sufficient mixing between buffer and colon tissue, then were place inside an anaerobic chamber for incubation. After one-hour the supernatant from each pouch was diluted, and plated onto BHIS agar plates plus Clostridium difficile selective supplement (Thermo Scientific, UK), which were incubated in the anaerobic chamber for 24 hours for CFU determination. Bacterial survival between the control and treated groups—were analyzed statistically by Students t-test using Prism.

### Results

Expression and Purification of PlyCD and PlyCD₁₋₁₇₄ from E. coli

The sequence of PlyCD and PlyCD₁₋₁₇₄ were separately cloned into a pBAD24 expression vector as described in the Methods. After arabinose induction, the whole-cell lysate of PlyCD or PlyCD₁₋₁₇₄, was purified by cation exchange chromatography. By SDS-PAGE, PlyCD exhibited a protein size of 28 kDa (FIG. 2a), and PlyCD₁₋₁₇₄ of 20 kDa (FIG. 2c). Eluted fractions containing the purified target protein were pooled and the final purified products revealed >90% purity for both molecules (FIGS. 2b and 2d). The average yield for both was about 6 mg protein per liter of E. coli culture. These purified proteins were used in all subsequent experiments.

Molecular Characterization of PlyCD: pH and Salt

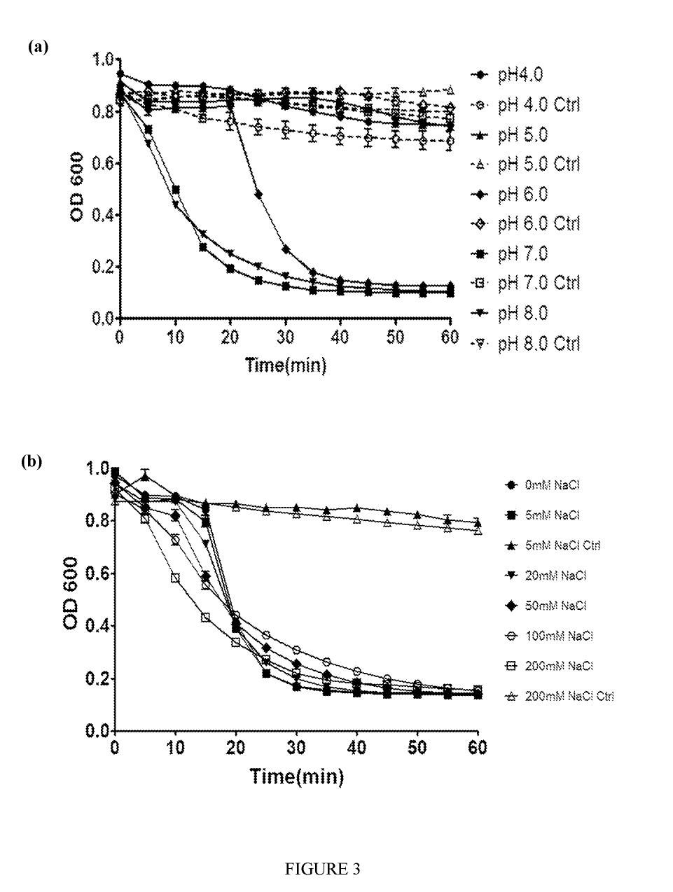

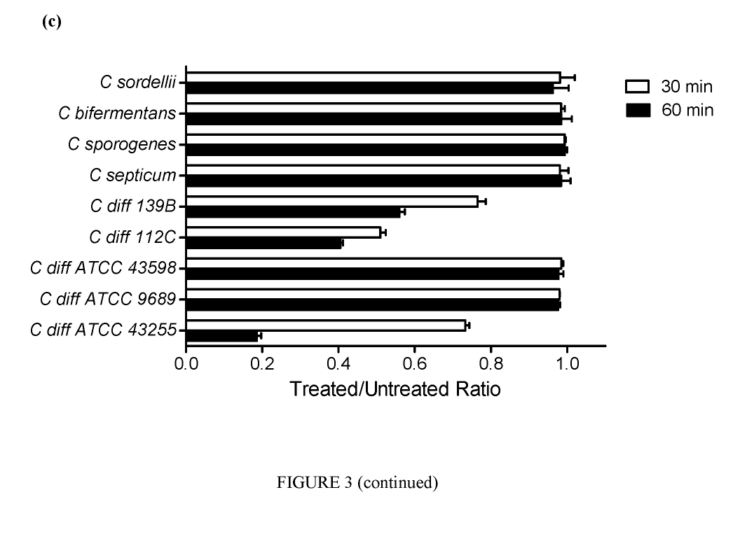

The activity of a lysin is often affected by salt concentration and pH. To investigate the lytic activity of PlyCD under varying conditions, the reduction in OD600 of Clostridium difficile suspensions was monitored over 60 mins. PlyCD displayed the strongest activity at pH 7.0 and pH 8.0 (FIG. 3a), and showed moderate activity at pH 6.0. At pH 4.0 and pH 5.0, it displayed no lytic activity. To test salt sensitivity, the lytic activity of PlyCD was measured under different NaCl concentrations. Data indicated that PlyCD exhibited maximum lytic activity in the absence of salt, and decreased in activity as NaCl concentration increased (FIG. 3b). At 400 mM NaCl, Clostridium difficile start to display autolysis, making analysis at or above this concentration unreliable (data not shown).

Specificity of Lytic Activity

Phage lysins generally exhibit high specificity [44], displaying elevated activity against a few closely related species, though lysins with broader activity do exist [33]. To study the specificity of PlyCD, we tested its lytic action against multiple Clostridium difficile strains and Clostridium species. All species were cultured anaerobically until mid-exponential phase, washed and resuspended in 20 mM PB, pH 7.0. PlyCD was added to the bacterial suspensions at a final concentration of 12.5 μM. OD600 values of each culture were recorded for 60 minutes. The ratio of OD600 at 30 and 60 minutes vs. time 0 was calculated. Of all the Clostridium species tested, PlyCD demonstrated the most effective lytic activity against Clostridium difficile strain ATCC-43255 and had moderate activity against two clinical strains 139B and 112C. Interestingly, the full length lysin did not have activity against the other Clostridium species tested or Clostridium difficile strains ATCC-9859 and ATCC-43593, suggesting PlyCD has a very specific and narrow range of activity against Clostridium difficile strains (FIG. 3c).

Molecular Characterization of PlyCD₁₋₁₇₄

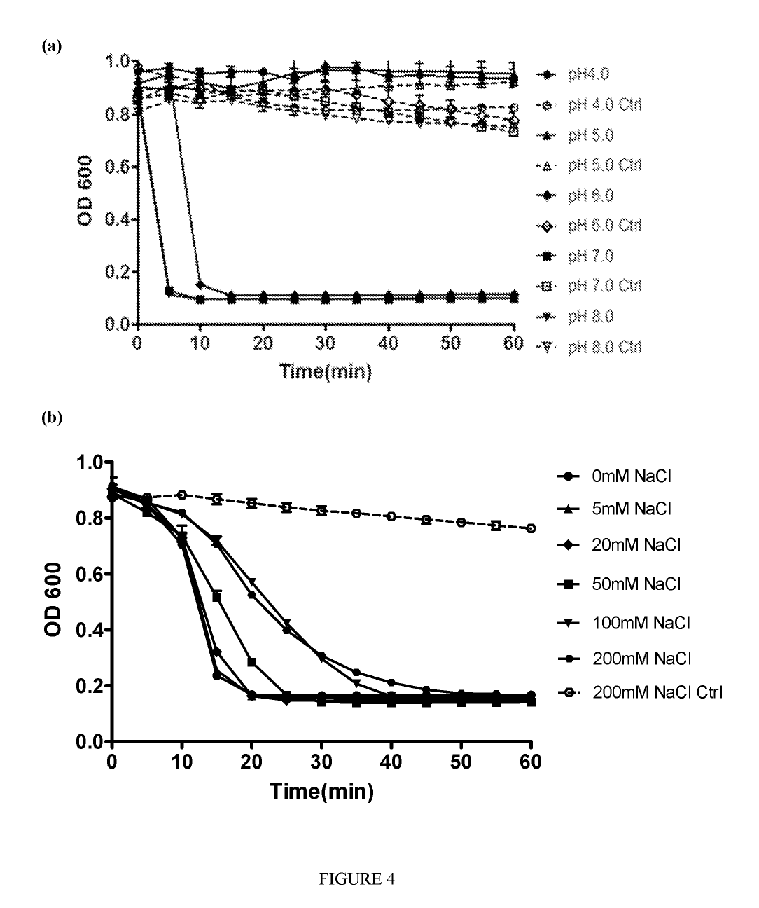

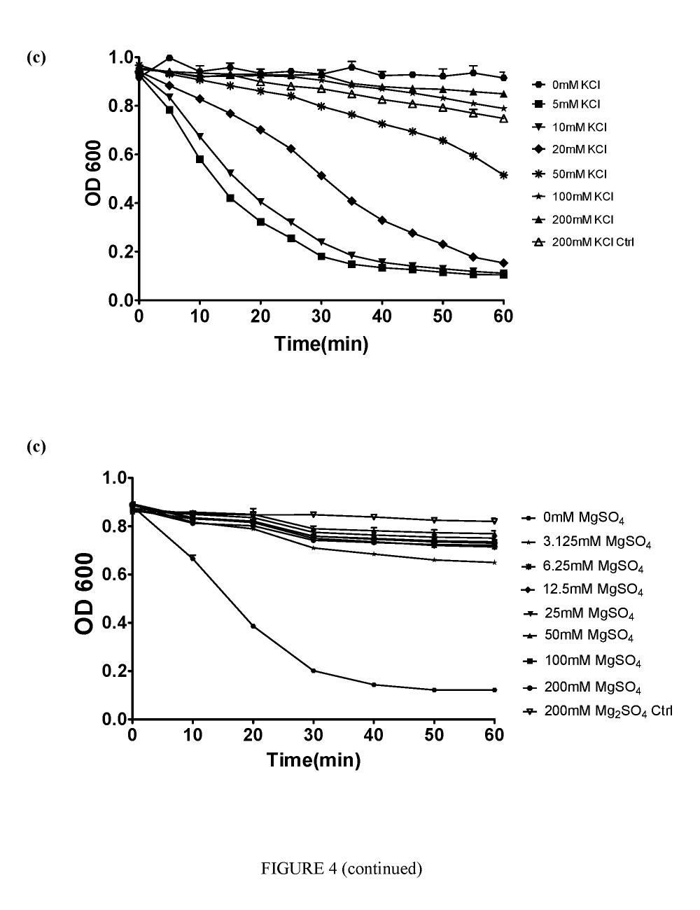

Similar to the intact PlyCD the lytic activity of PlyCD₁₋₁₇₄ also displayed very effective lytic efficiency at neutral to basic pH, pH 6.0, pH 7.0 and pH 8.0 (FIG. 4a), and no activity at pH 4.0 and pH 5.0. The salt sensitivity of PlyCD₁₋₁₇₄, was also similar to PlyCD where PlyCD₁₋₁₇₄ had maximum lytic activity in the absence of salt (FIG. 4b). A similar pattern was also observed in the presence of KCl (FIG. 4c). For subsequent experiments, PlyCD₁₋₁₇₄ was used in 20 mM PB at pH 7.0.

The lytic activity of PlyCD₁₋₁₇₄ was compared to PlyCD using a change in OD600 of a Clostridium difficile suspension over 60 minutes. While the OD600 value of the buffer control did not change in 60 minutes, the OD600 of PlyCD₁₋₁₇₄ dropped significantly and quickly when 12.5 μM (50 lag) was added to the bacterial cells (FIG. 5a), and within 20 minutes, the OD600 value dropped to baseline. In contrast, PlyCD-treated samples displayed a lower lytic activity, with only ^{˜}30% decrease after 30 minutes compared to the initial OD value, and the baseline was not reached until ^{˜}60 minutes. This comparison showed that PlyCD₁₋₁₇₄ has a greater lytic activity than full-length PlyCD, therefore, PlyCD₁₋₁₇₄ was chosen for subsequent experiments.

To further evaluate the lytic activity of the catalytic domain, various concentrations of PlyCD₁₋₁₇₄, were mixed with Clostridium difficile ATCC 43255 and the OD600 was monitored over 60 minutes. The results reveal that the lytic activity of PlyCD₁₋₁₇₄ functions in a dose-dependent manner (FIG. 5b). To determine the direct bactericidal activity of PlyCD₁₋₁₇₄ on Clostridium difficile cells, 12.5 uM of PlyCD₁₋₁₇₄ was mixed with 10⁸/mL Clostridium difficile ATCC 43255 cells in PB. The cell suspension was incubated anaerobically for 30 and 60 minutes and at each time point the surviving bacteria were plated for enumeration. After 30 and 60 minutes of lysin exposure, a 3 and 4-log drop respectively in Clostridium difficile survival was observed (FIG. 5c).

Specificity of PlyCD₁₋₁₇₄

PlyCD₁₋₁₇₄ was tested against a variety of Clostridium difficile strains, other Clostridia, and non-Clostridium-like species. All species were cultured until mid-exponential phase, washed and resuspended in 20 mM PB, pH 7.0. PlyCD₁₋₁₇₄ was added to the buffer at a final concentration of 12.51 μM. Unlike the full-length molecule, PlyCD₁₋₁₇₄ displayed strong activity against all of the Clostridium difficile laboratory strains, ATCC-43255, ATCC-9689, and ATCC-43583, as well as the two clinical strains, 139B and 112C, but not against other Clostridium species tested with the exception of C. sordelli (FIG. 6A). This indicates that PlyCD₁₋₁₇₄ has a better lytic activity and wider Clostridium difficile strain spectrum compared to PlyCD, yet it is not active against other potential intestinal Clostridium species, such as C. novyi, C. perfringens, C. bifermentans, C. sporogenes, and C. septicum. To determine if PlyCD₁₋₁₇₄ is effective against other non-Clostridium species, strains of Enterococcus, Staphylococcus, Streptococcus, Bacillus, Lactobacillus, and Listeria were also tested. PlyCD₁₋₁₇₄ did not display lytic activity, by OD600 drop, against any of these species, except for B. subtilis (FIG. 6B). These data suggest that PlyCD₁₋₁₇₄, compared to PlyCD, was more active against many Clostridium difficile strains while still retaining its specificity.

Protection Against Clostridium difficile Infection In Vivo

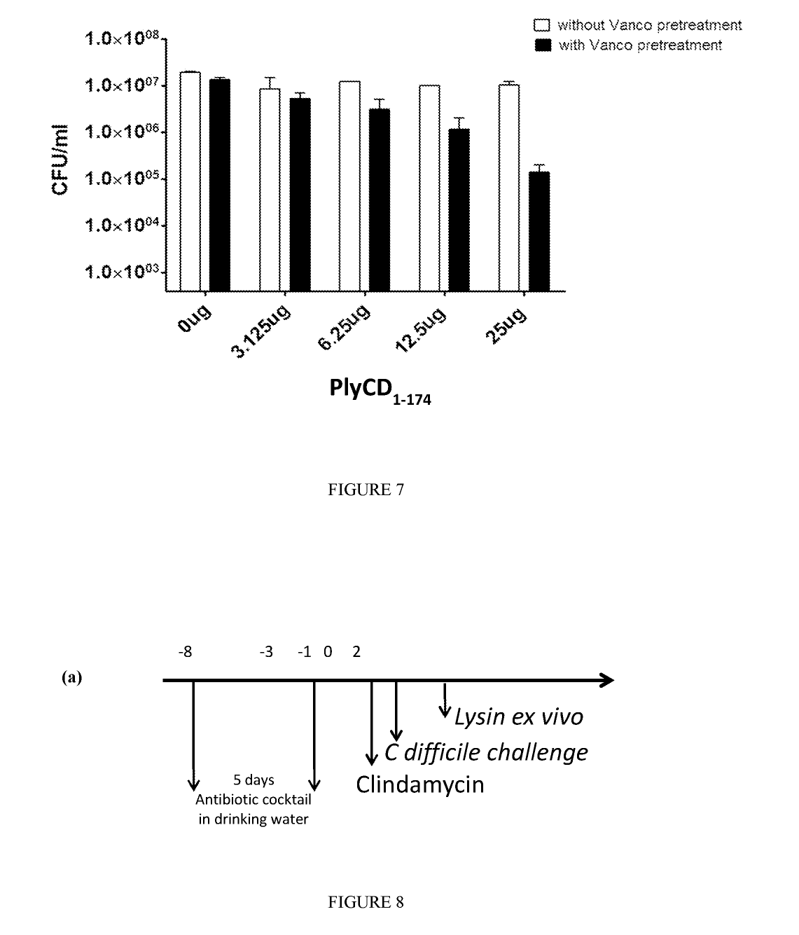

To assess whether PlyCD₁₋₁₇₄ could protect against Clostridium difficile infection in vivo, we established a Clostridium difficile infection model in mice, adapted from a previously described model [41]. Mice were administered 5 days of antibiotic cocktail in their drinking water, followed by 2 days of autoclaved regular water, then an intraperitoneal injection of clindamycin the day before infection. The mice were then fed with 2×10⁶ Clostridium difficile spores via gavage and monitored for symptoms (FIG. 7A). The majority of Clostridium difficile infected mice started to develop visible diarrhea one to two days after infection, as judged by the loose stools on the cage floor and wet perianal region and tails. Mice received 250 μL of PlyCD₁₋₁₇₄ (400 ug/mouse) or PB control via intra-rectal injection through a 20-gauge tube inserted 3.5 cm into the colon at 24 and 48 hours post infection. PB-treated mice became moribund at day 3 post infection, with 6 dead on day 3, which resulted in a final survival rate of 20%. Alternatively, mice treated with the PlyCD₁₋₁₇₄ enema had a 60% survival rate by day 3, with only 3 dead on day 3 and 1 dead on day 4, respectively (FIG. 7B). These results show that PlyCD₁₋₁₇₄ can be protective when treating Clostridium difficile infection in mice. Even better results were possibly confounded by the nature of the mouse model of Clostridium difficile infection. For example, necropsy of the dead mice revealed some solid stool still remaining in the intestines, which may have prevented complete distribution of the PlyCD₁₋₁₇₄.

Reduction of Clostridium difficile Bacteria in an Ex Vivo Mouse Colon Infection Model

We modified the in vivo infection model to develop an ex vivo mouse treatment model. In this model we asked whether the PlyCD₁₋₁₇₄ lysin was able to function and kill Clostridium difficile bacteria in the colonic environment and whether there were any inhibitory substances that would block its activity. Two days after C. difficile infection, large intestines from mice with advanced infection were excised between the cecum and anus and divided into two pieces to be treated with either PlyCD₁₋₁₇₄ or PB alone (FIG. 8A). In a 500-μl total reaction volume, 600 μg of PlyCD₁₋₁₇₄ decreased the titer of intestinal C.difficile from an average of 6.5×10⁶ CFU/ml to 5.2×10⁴ CFU per mL and 4.5×10⁴ CFU/ml (2 log reduction) after 30 or 60 min of incubation, respectively (FIG. 8B). Since the intestinal volume between the cecum and anus of a 6- to 8-week-old mouse is about 250 μL, we repeated the experiment by adding 250 μL PB to the intestinal pieces with or without 600 μg PlyCD₁₋₁₇₄. Intestines that were treated with 250 μl of PlyCD₁₋₁₇₄ also displayed an approximate two-log decrease in C. difficile CFU after 60 min of incubation (FIG. 8C). These data suggest that PlyCD₁₋₁₇₄ is active against Clostridium difficile vegetative cells in the presence of the large intestine and its contents.

The Immunofluorescence Imaging of PlyCD Binding Domain

To better understand the binding characteristics of PlyCD, cells of C. difficile were fixed onto microscope slides and reacted with rhodamine labeled PlyCD. Binding of the labeled PlyCD to the bacteria was visualized by fluorescence microscopy. In the PlyCD-treated sample, some of C. difficile cells remained intact, while others appeared lysed, with the labeled PlyCD binding only to the lysed cells. No binding signal was observed on any of the cells that were still intact (FIG. 9). This suggests that PlyCD binds better to lysed C. difficile cells, and that the binding site on the cell wall of C. difficile is not fully accessible to the intact PlyCD molecule.

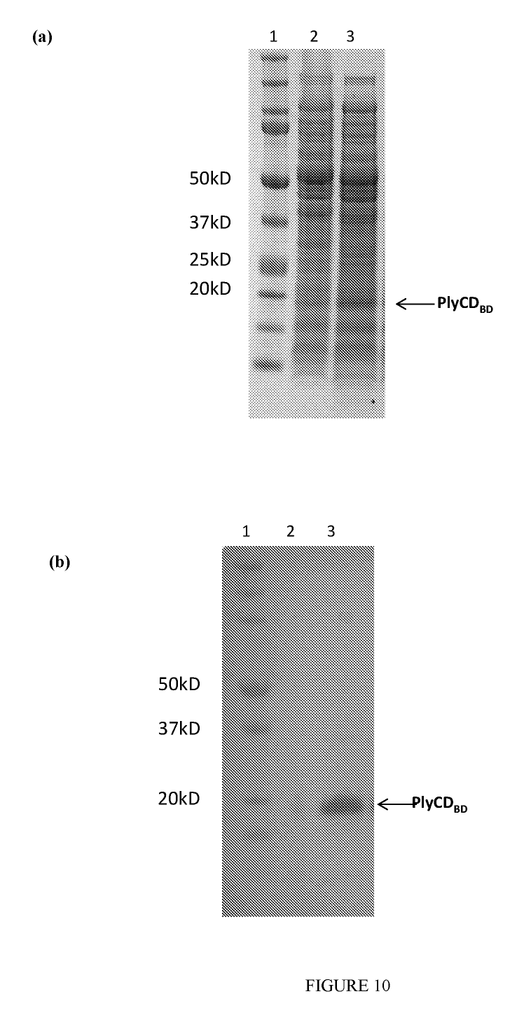

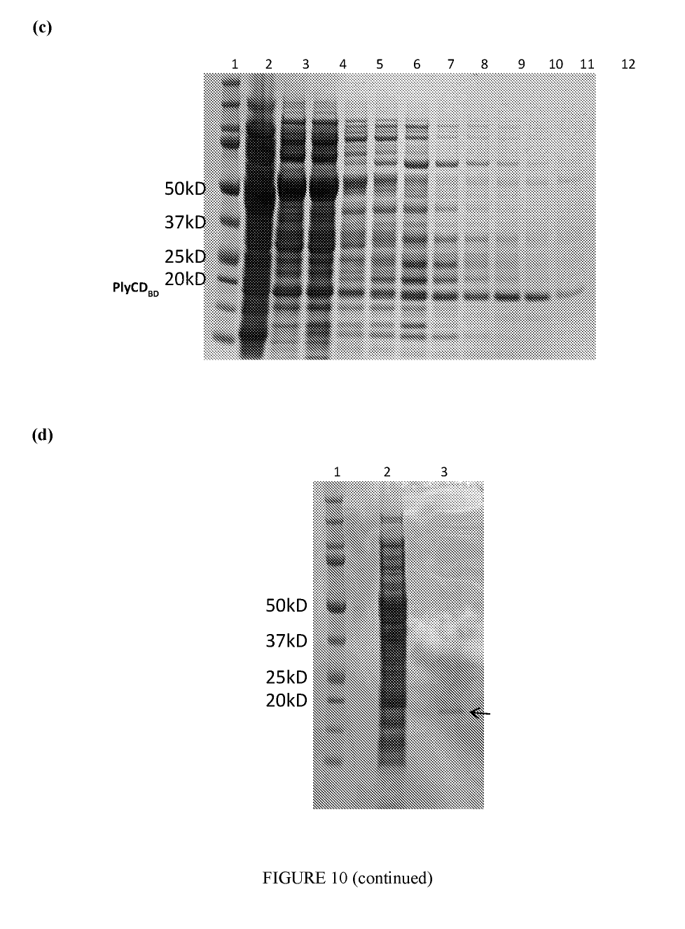

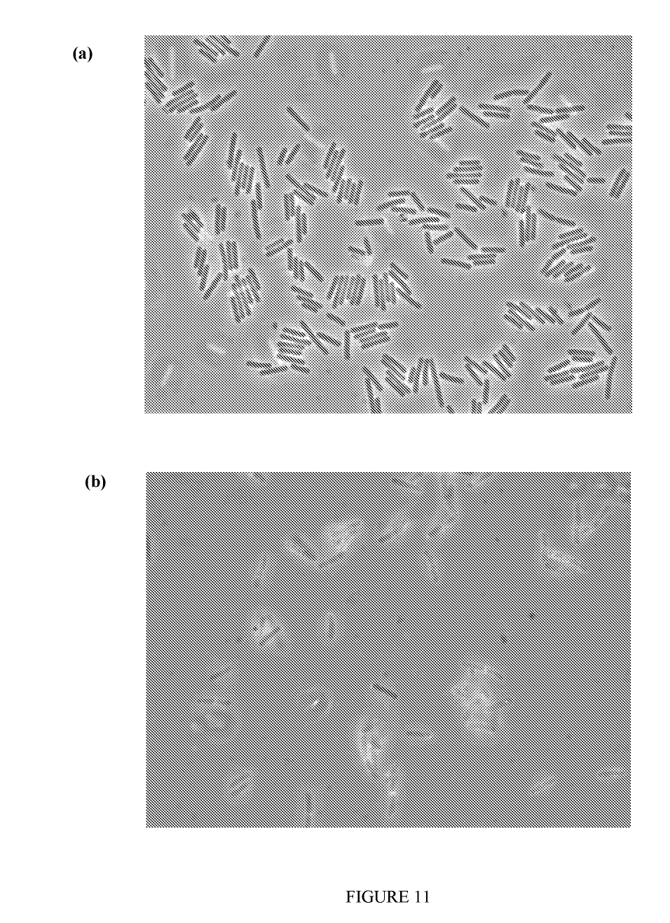

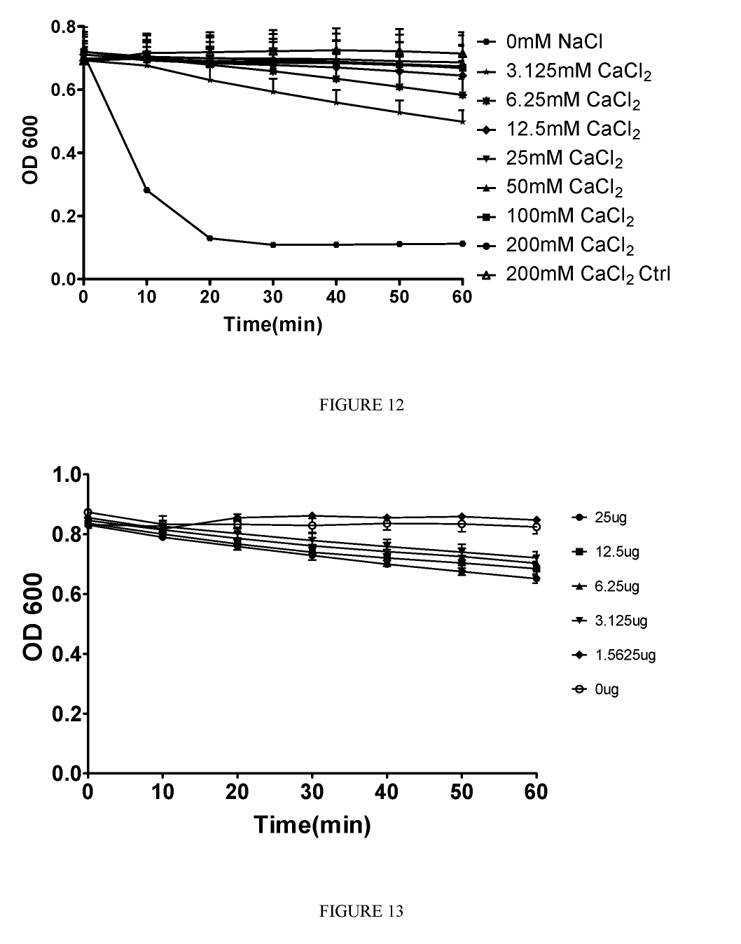

To further verify this observation, we expressed the C-terminal binding domain of PlyCD (PlyCDBD) with its N-terminal fused to a HIS-tag and double Etags in tandem. Arabinose induced expression of PlyCDBD (FIG. 10A), was verified though immunoblotting via an Etag antibody (FIG. 10B). The induced PlyCD lysate was purified on a Nickel column via its HIS-tag (FIG. 10C), and the final purified product was found to be homogeneous (FIG. 10D). The binding of PlyCDBD to cell wall was visualized using immunofluorescence microscopy by FITC-conjugated Etag antibody. When applying PlyCDBD directly to the mounted C. difficile cells, the majority of cells remain intact. There was not any binding of PlyCDBD to the intact cell walls (FIG. 11A). However, when the cells were pretreated with PlyCD₁₋₁₇₄, a majority of the cells lysed and PlyCDBD was able to bind specifically to the degraded cell walls of those cells (FIG. 11B). These results are consistent with PlyCD₁₋₁₇₄ displaying stronger lytic activity compared to PlyCD as determined by OD drop. Due to its smaller molecular size compared to PlyCD, the catalytic domain PlyCD₁₋₁₇₄, could potentially access substrates on the cell wall more readily. The difference in substrate accessibility between PlyCD₁₋₁₇₄ and PlyCD might play a role in the in lytic efficiency of these lysins.

This invention may be embodied in other forms or carried out in other ways without departing from the spirit or essential characteristics thereof. The present disclosure is therefore to be considered as in all aspects illustrative and not restrictive, the scope of the invention being indicated by the appended Claims, and all changes, which come within the meaning and range of equivalency are intended to be embraced therein.

Various references are cited throughout this Specification, each of which is incorporated herein by reference in its entirety.

The citation of references herein shall not be construed as an admission that such is prior art to the present invention.

### REFERENCES (ADDITIONAL REFERENCES IN IN-TEXT CITATIONS ABOVE)

1. Heinlen L, Ballard J D (2010) Clostridium difficile infection. Am J Med Sci 340: 247-252.
2. CDC (2015) www.cdc.gov/HAI/organisms/cdiff/Cdiff_infect.html.
3. Bartlett J G (2006) Narrative review: the new epidemic of Clostridium difficile-associated enteric disease. Ann Intern Med 145: 758-764.
4. Elliott B, Chang B J, Golledge C L, Riley T V (2007) Clostridium difficile-associated diarrhoea. Intern Med J 37: 561-568.
5. Loo V G, Poirier L, Miller M A, Oughton M, Libman M D, et al. (2005) A predominantly clonal multi-institutional outbreak of Clostridium difficile-associated diarrhea with high morbidity and mortality. N Engl J Med 353: 2442-2449.
6. MacCannell D R, Louie T J, Gregson D B, Laverdiere M, Labbe A C, et al. (2006) Molecular analysis of Clostridium difficile PCR ribotype 027 isolates from Eastern and Western Canada. J Clin Microbiol 44: 2147-2152.
7. Warny M, Pepin J, Fang A, Killgore G, Thompson A, et al. (2005) Toxin production by an emerging strain of Clostridium difficile associated with outbreaks of severe disease in North America and Europe. Lancet 366: 1079-1084.
8. Leffler D A, Lamont J T (2009) Treatment of Clostridium difficile-associated disease. Gastroenterology 136: 1899-1912.
9. Monaghan T, Boswell T, Mahida Y R (2008) Recent advances in Clostridium difficile-associated disease. Gut 57: 850-860.
10. Pepin J, Alary M E, Valiquette L, Raiche E, Ruel J, et al. (2005) Increasing risk of relapse after treatment of Clostridium difficile colitis in Quebec, Canada. Clin Infect Dis 40: 1591-1597.
11. Surawicz C M, Alexander J (2011) Treatment of refractory and recurrent Clostridium difficile infection. Nat Rev Gastroenterol Hepatol 8: 330-339.
12. Cohen S H, Gerding D N, Johnson S, Kelly C P, Loo V G, et al. (2010) Clinical practice guidelines for Clostridium difficile infection in adults: 2010 update by the society for healthcare epidemiology of America (SHEA) and the infectious diseases society of America (IDSA). Infect Control Hosp Epidemiol 31: 431-455.
13. Barbut F, Richard A, Hamadi K, Chomette V, Burghoffer B, et al. (2000) Epidemiology of recurrences or reinfections of Clostridium difficile-associated diarrhea. J Clin Microbiol 38: 2386-2388.
14. Alzahrani N, Johani S A (2013) Emergence of a highly resistant Clostridium difficile strain (NAP/BI/027) in a tertiary care center in Saudi Arabia. Ann Saudi Med 33: 198-199.
15. Huang J S, Jiang Z D, Garey K W, Lasco T, Dupont H L (2013) Use of rifamycin drugs and development of infection by rifamycin-resistant strains of Clostridium difficile. Antimicrob Agents Chemother 57: 2690-2693.
16. Rohlke F, Stollman N (2012) Fecal microbiota transplantation in relapsing Clostridium difficile infection. Therap Adv Gastroenterol 5: 403-420.
17. Agito M D, Atreja A, Rizk M K (2013) Fecal microbiota transplantation for recurrent C difficile infection: ready for prime time? Cleve Clin J Med 80: 101-108.
18. Hell M, Bernhofer C, Stalzer P, Kern J M, Claassen E (2013) Probiotics in Clostridium difficile infection: reviewing the need for a multistrain probiotic. Benef Microbes 4: 39-51.
19. O'Horo J C, Jindai K, Kunzer B, Safdar N (2014) Treatment of recurrent Clostridium difficile infection: a systematic review. Infection 42: 43-59.
20. Parisien A, Allain B, Zhang J, Mandeville R, Lan C Q (2008) Novel alternatives to antibiotics: bacteriophages, bacterial cell wall hydrolases, and antimicrobial peptides. J Appl Microbiol 104: 1-13.
21. Petty N K, Evans T J, Fineran P C, Salmond G P (2007) Biotechnological exploitation of bacteriophage research. Trends Biotechnol 25: 7-15.
22. Meader E, Mayer M J, Gasson M J, Steverding D, Carding S R, et al. (2010) Bacteriophage treatment significantly reduces viable Clostridium difficile and prevents toxin production in an in vitro model system. Anaerobe 16: 549-554.
23. Goh S, Riley T V, Chang B J (2005) Isolation and characterization of temperate bacteriophages of Clostridium difficile. Appl Environ Microbiol 71: 1079-1083.
24. Goh S, Ong P F, Song K P, Riley T V, Chang B J (2007) The complete genome sequence of Clostridium difficile phage phiC2 and comparisons to phiCD119 and inducible prophages of CD630. Microbiology 153: 676-685.
25. Govind R, Fralick J A, Rolfe R D (2006) Genomic organization and molecular characterization of Clostridium difficile bacteriophage PhiCD119. J Bacteriol 188: 2568-2577.
26. Mayer M J, Narbad A, Gasson M J (2008) Molecular characterization of a Clostridium difficile bacteriophage and its cloned biologically active endolysin. J Bacteriol 190: 6734-6740.
27. Horgan M, O'Sullivan O, Coffey A, Fitzgerald G F, van Sinderen D, et al. (2010) Genome analysis of the Clostridium difficile phage PhiCD6356, a temperate phage of the Siphoviridae family. Gene 462: 34-43.
28. Mahony D E, Bell P D, Easterbrook K B (1985) Two bacteriophages of Clostridium difficile. J Clin Microbiol 21: 251-254.
29. Nagy E, Foldes J (1991) Electron microscopic investigation of lysogeny of Clostridium difficile strains isolated from antibiotic-associated diarrhea cases and from healthy carriers. APMIS 99: 321-326.
30. Fischetti V A (2005) Bacteriophage lytic enzymes: novel anti-infectives. Trends Microbiol 13: 491-496.
31. Nelson D, Schuch R, Chahales P, Zhu S, Fischetti V A (2006) PlyC: a multimeric bacteriophage lysin. Proc Natl Acad Sci USA 103: 10765-10770.
32. Daniel A, Euler C, Collin M, Chahales P, Gorelick K J, et al. (2010) Synergism between a novel chimeric lysin and oxacillin protects against infection by methicillin-resistant Staphylococcus aureus. Antimicrob Agents Chemother 54: 1603-1612.
33. Gilmer D B, Schmitz J E, Euler C W, Fischetti V A (2013) Novel bacteriophage lysin with broad lytic activity protects against mixed infection by Streptococcus pyogenes and methicillin-resistant Staphylococcus aureus. Antimicrob Agents Chemother 57: 2743-2750.
34. Schuch R, Pelzek A J, Raz A, Euler C W, Ryan P A, et al. (2013) Use of a bacteriophage lysin to identify a novel target for antimicrobial development. PLoS One 8: e60754.
35. Fenton M, Ross R P, McAuliffe O, O'Mahony J, Coffey A (2011) Characterization of the staphylococcal bacteriophage lysin CHAP(K). J Appl Microbiol 111: 1025-1035.
36. Resch G, Moreillon P, Fischetti V A (2011) A stable phage lysin (Cpl-1) dimer with increased antipneumococcal activity and decreased plasma clearance. Int J Antimicrob Agents 38: 516-521.
37. Pastagia M, Euler C, Chahales P, Fuentes-Duculan J, Krueger J G, et al. (2011) A novel chimeric lysin shows superiority to mupirocin for skin decolonization of methicillin-resistant and -sensitive Staphylococcus aureus strains. Antimicrob Agents Chemother 55: 738-744.
38. Schuch R, Nelson D, Fischetti V A (2002) A bacteriolytic agent that detects and kills Bacillus anthracis. Nature 418: 884-889.
39. Loessner M J (2005) Bacteriophage endolysins—current state of research and applications. Curr Opin Microbiol 8: 480-487.
40. Sun X, Wang H, Zhang Y, Chen K, Davis B, et al. (2011) Mouse relapse model of Clostridium difficile infection. Infect Immun 79: 2856-2864.
41. Chen X, Katchar K, Goldsmith J D, Nanthakumar N, Cheknis A, et al. (2008) A mouse model of Clostridium difficile-associated disease. Gastroenterology 135: 1984-1992.
42. Raz A, Fischetti V A (2008) Sortase A localizes to distinct foci on the Streptococcus pyogenes membrane. Proc Natl Acad Sci USA 105: 18549-18554.
43. Yoong P, Schuch R, Nelson D, Fischetti V A (2004) Identification of a broadly active phage lytic enzyme with lethal activity against antibiotic-resistant Enterococcus faecalis and Enterococcus faecium. J Bacteriol 186: 4808-4812.
44. Fischetti V A (2008) Bacteriophage lysins as effective antibacterials. Curr Opin Microbiol 11: 393-400.
45. Nelson D, Loomis L, Fischetti V A (2001) Prevention and elimination of upper respiratory colonization of mice by group A streptococci by using a bacteriophage lytic enzyme. Proc Natl Acad Sci USA 98: 4107-4112.
46. Lood R, Raz A, Molina H, Euler C W, Fischetti V A (2014) A highly active and negatively charged Streptococcus pyogenes lysin with a rare D-alanyl-L-alanine endopeptidase activity protects mice against streptococcal bacteremia. Antimicrob Agents Chemother.
47. Porter C J, Schuch R, Pelzek A J, Buckle A M, McGowan S, et al. (2007) The 1.6 A crystal structure of the catalytic domain of PlyB, a bacteriophage lysin active against Bacillus anthracis. J Mol Biol 366: 540-550.
48. Low L Y, Yang C, Perego M, Osterman A, Liddington R C (2005) Structure and lytic activity of a Bacillus anthracis prophage endolysin. J Biol Chem 280: 35433-35439.
49. Meroueh S O, Bencze K Z, Hesek D, Lee M, Fisher J F, et al. (2006) Three-dimensional structure of the bacterial cell wall peptidoglycan. Proc Natl Acad Sci USA 103: 4404-4409.
50. Low L Y, Yang C, Perego M, Osterman A, Liddington R (2011) Role of net charge on catalytic domain and influence of cell wall binding domain on bactericidal activity, specificity, and host range of phage lysins. J Biol Chem 286: 34391-34403.
51. Pawlowski S W, Calabrese G, Kolling G L, Platts-Mills J, Freire R, et al. (2010) Murine model of Clostridium difficile infection with aged gnotobiotic C57BL/6 mice and a BI/NAP1 strain. J Infect Dis 202: 1708-1712.
52. Theriot C M, Koumpouras C C, Carlson P E, Bergin, I I, Aronoff D M, et al. (2011) Cefoperazone-treated mice as an experimental platform to assess differential virulence of Clostridium difficile strains. Gut Microbes 2: 326-334.
53. Burke K E, Lamont J T (2013) Fecal transplantation for recurrent Clostridium difficile infection in older adults: a review. J Am Geriatr Soc 61: 1394-1398.
54. van Nood E, Vrieze A, Nieuwdorp M, Fuentes S, Zoetendal E G, et al. (2013) Duodenal infusion of donor feces for recurrent Clostridium difficile. N Engl J Med 368: 407-415.
55. Chen X, Katchar K, Goldsmith J D, Nanthakumar N, Cheknis A, Gerding D N, Kelly C P. 2008. A mouse model of Clostridium difficile-associated disease. Gastroenterology 135:1984-1992.
56. PLoS Pathogens (2014) 10(7):e1004228.

1. A method for reducing a population of bacteria comprising gram positive bacteria, the method comprising contacting the bacteria with a composition comprising a lytic enzyme that comprises the amino acid sequence of SEQ ID NO: 2 or an amino acid sequence with a segment having at least 95% identity to the amino acid sequence of SEQ ID NO:2 such that gram positive bacteria in the population are killed.

2. The method of claim 1, wherein the lytic enzyme does not comprise SEQ ID NO:3.

3. The method of claim 1, wherein the lytic enzyme consists of the sequence of SEQ ID NO:2.

4. The method of any one of claims 1-3 wherein the gram positive bacteria in the population that are killed comprise Clostridium difficile.

5. The method of claim 4, wherein the gram positive bacteria in the population that are killed further comprise Clostridium sordellii and/or Bacillus subtilis.

6. The method of claim 4, wherein the Clostridium difficile comprise antibiotic-resistant Clostridium difficile.

7. The method of claim 4, wherein the gram positive bacteria in the population that are killed are in an individual.

8. The method of claim 7, wherein the population of bacteria in the individual further comprise commensal bacteria, wherein the commensal bacteria are not killed by the lytic enzyme.

9. The method of claim 8, wherein the commensal bacteria are selected from C. septicum, C. novyi, E. faecalis, E. faecium, L. rhamnosous, and combinations thereof.

10. A method for prophylaxis and/or treatment of an individual exposed to or at risk for exposure to a pathogenic Clostridium difficile bacteria comprising administering to the individual a composition comprising a lytic enzyme that comprises the amino acid sequence of SEQ ID NO: 2 or an amino acid sequence having a segment with at least 95% identity to the amino acid sequence of SEQ ID NO:2 in an amount effective to kill at least some of the Clostridium difficile bacteria.

11. The method of claim 10, wherein the lytic enzyme does not comprise SEQ ID NO:3.

12. The method of claim 10, wherein the lytic enzyme consists of the sequence of SEQ ID NO:2.

13. The method of any one of claims 10-12 wherein the subject is exposed to or at risk of infection by the Clostridium difficile bacteria and wherein the administering prevents or inhibits development of the infection.

14. The method of any one of claims 10-12, wherein the administering comprises administering the composition to the gastrointestinal system of the individual.

15. The method of any one of claims 10-12, wherein the administering comprises contacting an external surface or skin of the individual with the composition.

16. A pharmaceutical composition for killing Clostridium difficile bacteria comprising a lytic enzyme that comprises the amino acid sequence of SEQ ID NO: 2 or an amino acid sequence with at least 95% identity to the amino acid sequence of SEQ ID NO:2, the composition further comprising at least one pharmaceutically acceptable carrier or excipient.

17. The pharmaceutical composition of claim 16, wherein the lytic enzyme does not comprise SEQ ID NO:3.

18. The pharmaceutical composition of claim 16, wherein the lytic enzyme consists of the sequence of SEQ ID NO:2.

19. The pharmaceutical composition of any one of claims 16-18 wherein the lytic enzyme is produced by an expression vector.

20. A polypeptide capable of killing Clostridium difficile bacteria comprising a lytic enzyme that comprises the amino acid sequence of SEQ ID NO: 2 or an amino acid sequence with at least 95% identity to the amino acid sequence of SEQ ID NO:2, wherein the lytic enzyme does not comprise SEQ ID NO:3.

21. The polypeptide of claim 20 consisting of the sequence of SEQ ID NO:2.

22. The polypeptide of claim 20 or claim 21, wherein the polypeptide is in physical association with a Clostridium difficile bacterium.

23. The polypeptide of claim 20 or claim 21, wherein the polypeptide is in physical association with a component of peptidoglycan present in a Clostridium difficile bacterium.

24. A method of making a recombinant polypeptide capable of killing Clostridium difficile bacteria comprising expressing the recombinant polypeptide in a population of cells comprising an expression vector that encodes and expresses the recombinant polypeptide, wherein the recombinant polypeptide comprises an amino acid sequence of SEQ ID NO: 2 or an amino acid sequence with at least 95% identity to the amino acid sequence of SEQ ID NO:2, and separating the recombinant polypeptide from the population of cells.

25. (canceled)

26. The method of claim 24, wherein recombinant polypeptide consists of SEQ ID NO:2.

27. An expression vector encoding a polypeptide of claim 20 or claim 21.

28. A bacteria comprising the expression vector of claim 27.

29. A vessel comprising the pharmaceutical composition of any one of claims 16-18.
